SEQUENCE LISTING (6 sequences)

SEQ ID NO: 1 - DNA, 9450 bp, Artificial Sequence
  Synthetic plasmid pMBXS323
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcagctcatccagttgccgccatcgacgttcaacgtctgggcggtgatgtagtcggcatcggccgacgcgaggaacagcgcggcgcccgtcaggtcgcccggcacgcccatgcggccgagcggcacggcttcaccgacgagccgcttcttctcgccgagcggccggttctcgtagcgcgcgaacagcgcatcgacctgctcccacatcggcgtgtcgaccacgcccggcgcgatgccgttcacgttgatccggtgcggcgcgagcgcgagcgcggccgactgcgtatagctgatcaccgcggccttggtcgcgcagtagtgcgaaacgagcgcctcgccgcgacggccggcctgcgacgacatgttgacgatcttgccgccgcgcccctgctcgaccatccgttgcgcaaccgcctgcatcaggaagaacagccctttcacgttgaccgagaacagccggtcgaacacgtcccaggattcatcgaggagcggacgcatgtcgaacagcgccgcgttgttgaacagaatgtcgacgccgccgaagcgctcgaccgccgtggcgacgatccgcgtgatgtcgtcgcgacgcgtgacgtcggccgtgacggccaccgcgcggcccgggttggcctcgatcagccgcgcgagcgagccgcctgccggcttcacgtcgacgagcacgcagcgcgcgccctcgtccagatagcgttgtgcgaccgcctcgccgatgccgcttgcggcgcccgtcaggatcgcgaccttgtcttccagtctcattttgccgcttggtatctgcattacaatgaaatgagcaaagactatgtgagtaacactggtcaacactaggagaaggcatcgagcaagatacgtatgtaaagagaagcaatatagtatcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtcca

SEQ ID NO: 2 - DNA, 884 bp, Artificial Sequence
  Synthetic DNA fragment of a portion of hsp70 intron and sdh
tacgtatcttgctcgatgccttctcctagtgttgaccagtgttactcacatagtctttgctcatttcattgtaatgcagataccaagcggcaaaatgagactggaagacaaggtcgcgatcctgacgggcgccgcaagcggcatcggcgaggcggtcgcacaacgctatctggacgagggcgcgcgctgcgtgctcgtcgacgtgaagccggcaggcggctcgctcgcgcggctgatcgaggccaacccgggccgcgcggtggccgtcacggccgacgtcacgcgtcgcgacgacatcacgcggatcgtcgccacggcggtcgagcgcttcggcggcgtcgacattctgttcaacaacgcggcgctgttcgacatgcgtccgctcctcgatgaatcctgggacgtgttcgaccggctgttctcggtcaacgtgaaagggctgttcttcctgatgcaggcggttgcgcaacggatggtcgagcaggggcgcggcggcaagatcgtcaacatgtcgtcgcaggccggccgtcgcggcgaggcgctcgtttcgcactactgcgcgaccaaggccgcggtgatcagctatacgcagtcggccgcgctcgcgctcgcgccgcaccggatcaacgtgaacggcatcgcgccgggcgtggtcgacacgccgatgtgggagcaggtcgatgcgctgttcgcgcgctacgagaaccggccgctcggcgagaagaagcggctcgtcggtgaagccgtgccgctcggccgcatgggcgtgccgggcgacctgacgggcgccgcgctgttcctcgcgtcggccgatgccgactacatcaccgcccagacgttgaacgtcgatggcggcaactggatgagctgactcgagtgaattc

SEQ ID NO: 3 - DNA, 20 bp, Artificial Sequence
  Synthetic KMB 206 primer
tcgcacaacgctatctggac

SEQ ID NO: 4 - DNA, 20 bp, Artificial Sequence
  Synthetic KMB 207 primer
gatgccgttcacgttgatcc

SEQ ID NO: 5 - DNA, 8650 bp, Artificial Sequence
  Synthetic plasmid pSDH.dicot
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagtcagctcatccagttgccgccatcgacgttcaacgtctgggcggtgatgtagtcggcatcggccgacgcgaggaacagcgcggcgcccgtcaggtcgcccggcacgcccatgcggccgagcggcacggcttcaccgacgagccgcttcttctcgccgagcggccggttctcgtagcgcgcgaacagcgcatcgacctgctcccacatcggcgtgtcgaccacgcccggcgcgatgccgttcacgttgatccggtgcggcgcgagcgcgagcgcggccgactgcgtatagctgatcaccgcggccttggtcgcgcagtagtgcgaaacgagcgcctcgccgcgacggccggcctgcgacgacatgttgacgatcttgccgccgcgcccctgctcgaccatccgttgcgcaaccgcctgcatcaggaagaacagccctttcacgttgaccgagaacagccggtcgaacacgtcccaggattcatcgaggagcggacgcatgtcgaacagcgccgcgttgttgaacagaatgtcgacgccgccgaagcgctcgaccgccgtggcgacgatccgcgtgatgtcgtcgcgacgcgtgacgtcggccgtgacggccaccgcgcggcccgggttggcctcgatcagccgcgcgagcgagccgcctgccggcttcacgtcgacgagcacgcagcgcgcgccctcgtccagatagcgttgtgcgaccgcctcgccgatgccgcttgcggcgcccgtcaggatcgcgaccttgtcttccagtctcattttctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 6 - DNA, 5635 bp, Artificial Sequence
  Synthetic plasmid pUCSDH
tgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacgtgaaaacctctgacacatgcagctcccggagacggtcacagcttgtctgaagcggatgccgggagcagacaagcccgtcagggcgcgtcagcgggtgtggcgggtgtcggggctggcttaactatgcggcatcagagcagattgtacagagtgcaccatatgcggtgtgaaataccgcacagatgcgtaaggagaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcatgactgcaattttagagagacgcgaaagcgaaagcctatggggtcgcttctgtaactggataactagcactgaaaaccgtctttacattggatggtttggtgttttgatgatccctaccttattgacggcaacttctgtatttattattgccttcattgctgctcctccagtagacattgatggtattcgtgaacctgtttcagggtctctactttacggaaacaatattatttccggtgccattattcctacttctgcagctataggtttacatttttacccaatctgggaagcggcatccgttgatgaatggttatacaacggtggtccttatgaactaattgttctacacttcttacttggcgtagcttgttacatgggtcgtgagtgggagcttagtttccgtctgggtatgcgaccttggattgctgttgcatattcagctcctgttgcagctgctaccgcagttttcttgatctacccaattggtcaaggaagtttttctgatggtatgcctctaggaatctctggtactttcaatttcatgattgtattccaggctgagcacaacatccttatgcacccatttcacatgttaggcgtagctggtgtattcggcggctccctattcagtgctatgcatggttccttggtaacttctagtttgatcagggaaaccacagaaaatgaatctgctaatgaaggttacagattcggtcaagaggaagaaacttataacatcgtagccgctcatggttattttggccgattgatcttccaatatgctagtttcaacaactctcgttcgttacacttcttcctagctgcttggcctgtagtaggtatctggtttaccgctttaggtatcagcactatggctttcaacctaaatggtttcaatttcaaccaatctgtagttgacagtcaaggccgtgtaattaatacttgggctgatatcattaaccgtgctaaccttggtatggaagttatgcatgaacgtaatgctcacaacttccctctagacctagctgctatcgaagctccatctacaaatggataagtcgacaagtgtttgcggccgcgagctcggactcgagtttggatccaatcgatacaagtgagttgtagggagggaaccatgagactggaagacaaggtcgcgatcctgacgggcgccgcaagcggcatcggcgaggcggtcgcacaacgctatctggacgagggcgcgcgctgcgtgctcgtcgatgtgaagccggcaggcggctcgctcgcgcggctgatcgaggccaacccgggccgcgcggtggccgtcacggccgacgtcacgcgtcgcgacgacatcacgcggatcgtcgccacggcggtcgagcgcttcggcggcgttgacattctgttcaacaacgcggcgctgttcgacatgcgtccgctcctcgatgaatcctgggacgtgttcgaccggctgttctcggtcaacgtgaaagggctgttcttcctgatgcaggcggttgcgcaacggatggtcgagcaggggcgcggcggcaagatcgtcaacatgtcgtcgcaggccggccgtcgcggcgaggcgctcgtttcgcactactgcgcgaccaaggccgcggtgatcagctatacgcagtcggccgcgctcgcgctcgcgccgcaccggatcaacgtgaacggcatcgcgccgggcgtggtcgatacgccgatgtgggagcaggtcgatgcgctgttcgcgcgctacgagaaccggccgctcggcgagaagaagcggctcgtcggtgaagccgtgccgctcggccgcatgggcgtgccgggcgacctgacgggcgccgcgctgttcctcgcgtcggccgatgccgactacatcaccgcccagacgttgaacgtcgatggcggcaactggatgagctgaatctaagccgaattgggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgattgagtattctattttgattttgtatttgtttaaaattgtagaaatagaacttgtttctcttcttgctaatgttactatatctttttgattttttttttccaaaaaaaaaatcaaattttgacttcttcttatctcttatctttgaatatctcttatctttgaaataataatatcattgaaataagaaagaagagctatattcgaacttgaatcttttgttttctaatttaaataatgtaaaaacggaatgtaagtaggcgagggggcggatgtagccaagtggatcaaggcagtggattgtgaatccaccatgcgcgggttcaattcccgtcgttcgcccataattactcctattttttttttttttgtaaaaacgaagaatttaattcgattttctctcctatttactacggcgacgaagaatcaaattatcactatatttattcctttttctacttcttcttccaagtgcaggataaccccaaggggttgtgggtttttttctaccaattggggctctcccttcaccacccccatggggatggtctacagggttcataactactcctcttactacaggacgcttacctagccaacgcttagatccggctctacccaaacttttctggttcaccccaacattccccacttgtccgactgttgctgagcagtttttggatatcaaacggacctccccagaaggtaattttaatgtggccgatttcccctcttttgcaatcagtttcgctacagcacccgctgctctagctaattgtccaccctttccaagtgtgatttctatgttatgtatggccgtgcctaagggcatatcggttgaagtagattcttcttttgatcaatcaaaaccccttcccaaactgtacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatatta

(19) United States
(12) Patent Application Publication
(10) Pub. No.: US 2011/0321190 A1
(43) Pub. Date: Dec. 29, 2011

(54) METHOD OF POSITIVE PLANT SELECTION USING SORBITOL DEHYDROGENASE

(75) Inventors: Chakradhar Akula, Madison, WI (US); Karen Bohmert-Tatarev, Brookline, MA (US); Nii Patterson, Chelmsford, MA (US); Kristi D. Snell, Belmont, MA (US)

(73) Assignee: Metabolix, Inc.

(21) Appl. No.: 13/223,575

(22) Filed: Sep. 1, 2011

Related U.S. Application Data
(63) Continuation of application No. PCT/US2010/026546, filed on Mar. 8, 2010.
(60) Provisional application No. 61/158,132, filed on Mar. 6, 2009.

(51) Int. Cl. A01H 1/06; C12N 15/82; A01H 5/00; C12N 5/10

(52) U.S. Cl. 800/278; 435/412; 435/414; 435/415; 435/416; 435/419; 435/320.1; 800/296; 800/298; 800/306; 800/312; 800/317.3; 800/320; 800/320.1; 800/320.2; 800/322

(57) ABSTRACT

Transgenic plants and methods of culturing them using sorbitol as a sole carbon source are provided. One embodiment provides a method and system for positively selecting transgenic plants carrying and expressing a gene of interest. The transgenic plants are engineered to express sorbitol dehydrogenase in an amount effective to allow the transgenic plant to grow using sorbitol as the sole carbon source. In a preferred embodiment, the plant to be transformed does not have endogenous sorbitol dehydrogenase activity. Representative plants that can be transformed, include but are not limited to members of the Brassica family, industrial oilseeds, Arabidopsis thaliana, algae, soybean, cottonseed, sunflower, palm, coconut, rice, safflower, peanut, mustards, silage corn, alfalfa, switchgrass, miscanthus, sorghum, tobacco, sugarcane and flax.

### FIELD OF THE INVENTION

The invention is generally related to the field of plant molecular biology, more particularly to methods and compositions for positively selecting transformed or transfected plants.

### BACKGROUND OF THE INVENTION

The productivity and yield of plant crops can be improved by adding one or more input traits such as insect resistance, drought tolerance, herbicide tolerance, and yield improvement. Plants are also a desirable host for the production of a range of output traits including modified vegetable oils, seeds with increase oil content, biomaterials, amino acids, modified lignins, modified starches, nutraceutical products, precursor molecules that can be used to make biofuels, or compounds that can be used directly as biofuels. The production of plants with improved input or novel output traits usually requires transforming the plant material with a plant transformation vector carrying an expression cassette for the trait(s) of interest. To successfully select transformed plant tissue from untransformed tissue, a separate expression cassette encoding a selectable marker is routinely used.

A range of selectable markers have been used for plant transformation including markers encoding antibiotic resistance or herbicide tolerance, markers imparting the plant the ability to utilize a novel carbon source for growth, and markers encoding enzymes capable of detoxifying a compound that inhibits growth (Miki, B. and S. McHugh, “Selectable Marker Genes” in Transgenic Plants: Applications, Alternatives and Biosafety.” Journal of Biotechnology 107: 193-232 (2004); Dunwell, J. M., Plant Biotechnol. 3: 371 (2005); Goldstein, D. A., et al., J. Appl. Microbiol., 99(1): 7-23 (2005)). Selectable marker genes that have been used in extensively in plants include the neomycin phosphotransferase gene nptII (U.S. Pat. No. 5,034,322 to Rogers, et al., U.S. Pat. No. 5,530,196 to Fraley, et al.), hygromycin resistance gene (U.S. Pat. No. 5,668,298 to Waldron), the bar gene encoding resistance to phosphinothricin (U.S. Pat. No. 5,276,268 to Strauch, et al.), the expression of aminoglycoside 3″-adenyltransferase (aadA) to confer spectinomycin resistance (U.S. Pat. No. 5,073,675 to Jones, et al.), the use of inhibition resistant 5-enolpyruvyl-3-phosphoshikimate synthetase (U.S. Pat. No. 4,535,060 to Comai) and methods for producing glyphosate tolerant plants (U.S. Pat. No. 5,463,175 to Barry, et al.; U.S. Pat. No. 7,045,684 to Held, et al.).

Methods of plant selection that do not use antibiotics or herbicides as a selective agent have been previously described and include expression of glucosamine-6-phosphate deaminase to inactivate glucosamine in plant selection medium (U.S. Pat. No. 6,444,878 to Donaldson, et al.) and a positive/negative system that utilizes D-amino acids (Erikson, O., et al., Nat Biotechnol, 22(4): 455-458 (2004)). Barone and Widholm (Plant Cell Reports 27(3): 509-517 (2008)) developed a feedback-insensitive anthranilate synthase α-subunit of tobacco (ASA2) as a negative selectable marker using the tryptophan analogues 4-methylindole (4MI) or 7-methyl-DL-tryptophan (7MT) as the selection agent. Tryptophan analogues are toxic since they are able to mimic the feedback effect of tryptophan on anthranilate synthase, therefore inhibiting tryptophan biosynthesis which causes tryptophan deficiency for protein biosynthesis. Plants expressing the feedback-insensitive anthranilate synthase α-subunit of tobacco (ASA2) are able to survive on the tryptophan analogues and can be selected for. EP 0 530 129 A1 to Finn, O. et al. describes a positive selection system which enables the transformed plants to outgrow the non-transformed lines by expressing a transgene encoding an enzyme that activates an inactive compound added to the growth media. U.S. Pat. No. 5,767,378 to Bojsen, et al. describes the use of mannose or xylose for the positive selection of transgenic plants. U.S. Pat. No. 6,924,145 to Jorsboe, et al. describes a selection method based on transforming cells sensitive to galactose toxicity with a gene encoding UDP-glucose dependent uridyl transferase. U.S. Pat. No. 7,005,561 Parrott, et al. describes conferring to plant cells the ability to metabolize arabitol, ribitol, raffinose, sucrose, mannitol, or combinations, and then selecting transformants by selecting those cells that can grow on media containing those compounds.

EP 0 820 518 and U.S. Pat. No. 6,143,562, both to Trulson, et al., disclose the use of two expression cassettes to transform a plant cell. One cassette contains a gene that encodes an enzyme that converts an “encrypted” carbon source into a carbon source that can support growth of the cell, while the second cassette contains the gene of interest. Candidate first genes include (i) phosphomannose isomerase, which converts mannose-6-phosphate into fructose-6-phosphate, and where the encrypted carbon source would be mannose, (ii) mannitol-1-oxidoreductase which converts mannitol into mannose, and where mannitol is the encrypted carbon source, or (iii) human L-iditol dehydrogenase (EC 1.1.1.14), which converts sorbitol into fructose, and where sorbitol is the encrypted carbon source. Experimental results are provided showing the transformation of tomato, melon and squash with the pmi gene (phosphomannose isomerase; EC 5.3.1.8) via an Agrobacterium tumifaciens vector, so that transformed plants can be identified by their ability to grow on mannose as a carbon source. Maize and oat cell suspensions were also assessed for their ability to grow in liquid media containing mannose, and it was found that growth of non-transformed cells was reduced, relative to their growth in medium containing sucrose. The examples show that tomato cells do not grow on mannose, mannitol, sorbitol, lactose, trehalose or salicin. For sorbitol, candidate enzymes for converting it to fructose are listed as L-iditol dehydrogenase (EC 1.1.1.14) or D-sorbitol 1-oxidoreductase (EC 1.1.00.24). No information or guidance is provided regarding which plants are incapable of using these carbon sources as the sole source of carbon.

While all of these methods in principle allow the selection of transformed from untransformed plant material, it is advantageous to employ a selection system that does not utilize a gene encoding herbicide tolerance or antibiotic resistance when engineering plants for field use due to concerns of potential unwanted gene dispersal. It is also advantageous to limit the use of herbicide tolerance or antibiotic resistance genes in food, feed or industrial oilseed or biomass crops (Goldstein, D. et al., J. Appl. Microbia, 99(1): 7-23 (2005)).

Thus, there is a need for methods and compositions for positive selection of transformed, transfected, or transgenic plants or plant cells.

There is also a need for methods and compositions for positive selection of transgenic plants using sorbitol as a carbon source.

There is also a need for vectors and constructs designed to allow positive selection of transgenic plants.

There is also a need for methods for using sorbitol selection for the production of transgenic plants providing improved input and/or output traits.

There is also a need for constructs designed for efficient expression of the sorbitol dehydrogenase gene and other input and/or output traits in monocotyledonous plants.

There is also a need for constructs designed for efficient expression of the sorbitol dehydrogenase gene and other input and/or output traits in dicotyledonous plants.

There is also a need for constructs designed for efficient expression of the sorbitol dehydrogenase gene and other input and/or output traits in algae.

### SUMMARY OF THE INVENTION

Transgenic plants and methods of culturing them using sorbitol as a sole carbon source are provided. One embodiment provides a method and system for positively selecting transgenic plants carrying and expressing any other gene of interest. The transgenic plants are engineered to express sorbitol dehydrogenase in an amount effective to allow the transgenic plant to grow using sorbitol as the sole carbon source. In a preferred embodiment, the plant to be transformed does not have endogenous sorbitol dehydrogenase activity or does not have sufficient endogenous sorbitol dehydrogenase activity to enable a reasonable growth rate in tissue culture using sorbitol as the sole source of carbon. Representative plants that can be transformed, include but are not limited to any plant having poor or no growth in tissue culture using sorbitol as the sole carbon source selected from: members of the Brassica family, industrial oilseeds, algae, soybean, cottonseed, sunflower, palm, coconut, safflower, peanut, mustards, silage corn, alfalfa, switchgrass, miscanthus, sorghum, rice, tobacco, sugarcane and flax.

The gene of interest can by any gene. Typically the gene of interest encodes a polypeptide that confers a desired trait to the transgenic plant. The polypeptide can alter the metabolism of the plant, for example providing drought resistance, temperature resistance, increased yield, increased root growth, improved nitrogen use efficiency etc. The transgene can encode polypeptides that can produce a biopolymer, such as a polyhydroxyalkanoate (PHA), a vegetable oil containing fatty acids with a desirable industrial or nutritional profile, or a nutraceutical compound.

One embodiment provides a method for positively selecting transformed plants or plant cells by transforming a plant or plant cell with a heterologous nucleic acid encoding a polypeptide having sorbitol dehydrogenase activity and at least a second transgene encoding a second polypeptide, wherein the transformed plant expresses an effective amount of the polypeptide having sorbitol dehydrogenase activity to grow using sorbitol as a sole carbon source and culturing the transgenic plant using sorbitol as the sole carbon source. It will be appreciated that the nucleus or plastid of a plant can be transformed with the heterologous nucleic acid.

Vectors and constructs are provided for producing the disclosed transgenic plants. A preferred vector includes the nucleic acid sequence according to SEQ ID NO:2 or a complement thereof.

### BRIEF DESCRIPTION OF THE DRAWINGS

FIGS. 1A-1D are a set of 4 photographs showing the proliferation of wild-type switchgrass (Panicum virgatum cv. ‘Alamo’) callus cultures, in the presence of various sugars (FIG. 1A: maltose; FIG. 1B: fructose; FIG. 1C: sorbitol and no sugar; FIG. 1D:). Note the reduced growth of cultures in the presence of sorbitol as a sole carbon source and in the absence of any carbon source.

FIG. 2 illustrates the schematic plasmid map of the plant transformation vector pMBXS323 for enhanced expression of sdh in monocots.

FIGS. 3a and 3b are two photographs showing regeneration of shoots from callus transformed with pMBXS323 after growth on medium supplemented with sorbitol (FIG. 3a) and 3 week old, fully developed putative transgenic plants with root and shoot (FIG. 3b).

FIG. 4 is a photograph of an agarose gel showing samples from PCR analysis of soil grown plants tested with primers KMB 206 & KMB 207 for the presence of the sdh gene. The expected band size for primer set KMB 206 & KMB 207 is 0.49 kb. Labels are as follows: MW, DNA molecular weight markers; −C, negative control; WT, wild-type plant; +C, positive control PCR reaction using plasmid pMBXS323. DNA fragment size (in kb) is shown to left of gel.

FIG. 5 is a diagram illustrating the schematic plasmid map for plant nuclear transformation vector pSDH.dicot for expression of sorbitol dehydrogenase in dicots.

FIG. 6 is a diagram illustrating the schematic plasmid map for plastid transformation vector pUCSDH.

### DETAILED DESCRIPTION OF THE INVENTION

### I. Definitions

Before describing the various embodiments, it is to be understood that the invention is not limited in its application to the details of construction and the arrangement of the components set forth in the following description. Other embodiments can be practiced or carried out in various ways. Also, it is to be understood that the phraseology and terminology employed herein is for the purpose of description and should not be regarded as limiting.

Unless otherwise indicated, this disclosure encompasses conventional techniques of plant breeding, immunology, molecular biology, microbiology, cell biology and recombinant DNA, which are within the skill of the art. See, e.g., Sambrook and Russell, Molecular Cloning: A Laboratory Manual, 3rd edition (2001); Current Protocols In Molecular Biology [(F. M. Ausubel, et al. eds., (1987)]; Plant Breeding: Principles and Prospects (Plant Breeding, Vol 1) M. D. Hayward, N. O. Bosemark, I. Romagosa; Chapman & Hall, (1993.); Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)], Harlow and Lane, eds. (1988) Antibodies, A Laboratory Manual, and Animal Cell Culture [R. I. Freshney, ed. (1987)].

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience., 1999; and Robert A. Meyers (ed.), Molecular Biology and Biotechnology, a Comprehensive Desk Reference, published by VCH Publishers, Inc., 1995; Ausubel et al. (1987) Current Protocols in Molecular Biology, Green Publishing; Sambrook and Russell. (2001) Molecular Cloning: A Laboratory Manual 3rd. edition.

To facilitate understanding of the disclosure, the following definitions are provided:

To “alter” the expression of a target gene in a plant cell means that the level of expression of the target gene in a plant cell after applying a disclosed method of is different from its expression in the cell before applying the method. To alter gene expression preferably means that the expression of the target gene in the plant is upregulated.

When referring to expression, “control sequences” refers to DNA sequences necessary for the expression of an operably linked coding sequence in a particular host organism. Eukaryotic cells, including plant cells are known to utilize promoters, polyadenylation signals, and enhancers.

The term “cell” refers to a membrane-bound biological unit capable of replication or division.

The term “construct” refers to a recombinant genetic molecule having one or more isolated polynucleotide sequences. Genetic constructs used for transgene expression in a host organism include in the 5′-3′ direction, a promoter sequence; a sequence encoding a gene of interest, for example sorbitol dehydrogenase; and a termination sequence. The construct may also include selectable marker gene(s), other regulatory elements for expression, as well as one or more additional expression cassettes for expression other genes of interest.

As used herein, the term “control element” or “regulatory element” are used interchangeably to mean sequences positioned within or adjacent to a promoter sequence so as to influence promoter activity. Control elements may be positive or negative control elements. Positive control elements require binding of a regulatory element for initiation of transcription. Many such positive and negative control elements are known. Where heterologous control elements are added to promoters to alter promoter activity as described herein, they are positioned within or adjacent to the promoter sequence so as to aid the promoter's regulated activity in expressing an operationally linked polynucleotide sequence.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter.

The term “presequence” refers to a nucleic acid sequence positioned upstream of a coding sequence of interest. A nucleic acid sequence or polynucleotide is “operably linked” when it is placed into a functional relationship with another nucleic acid sequence. For example, DNA for a presequence or targeting sequence is operably linked to DNA for a polypeptide if it is expressed as a preprotein that participates in the targeting of the polypeptide to a subcellular compartment for example a plant plastid; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a presequence or targeting sequence, contiguous and in reading frame. Linking can be accomplished by ligation at convenient restriction sites. If such sites do not exist, synthetic oligonucleotide adaptors, linkers or gene synthesis are used in accordance with conventional practice.

The term “plant” is used in it broadest sense. It includes, but is not limited to, any species of woody, ornamental or decorative, crop or cereal, fruit or vegetable plant, and photosynthetic green algae (e.g., Chlamydomonas reinhardtii). It also refers to a plurality of plant cells that are largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

A non-naturally occurring plant refers to a plant that does not occur in nature without human intervention. Non-naturally occurring plants include transgenic plants and plants produced by non-transgenic means such as plant breeding.

The term “plant cell” refers to a structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “plant material” refers to leaves, stems, roots, flowers or flower parts, fruits, pollen, egg cells, zygotes, seeds, cuttings, cell or tissue cultures, or any other part or product of a plant.

A “plant organ” refers to a distinct and visibly structured and differentiated part of a plant such as a root, stem, leaf, flower bud, or embryo.

“Plant tissue” refers to a group of plant cells organized into a structural and functional unit. Any tissue of a plant whether in a plant or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

“Plasmids” are designated by a lower case “p” preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the inventive polynucleotides. Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

“Transformed,” “transgenic,” “transfected” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

A “transformed cell” refers to a cell into which has been introduced a nucleic acid molecule, for example by molecular biology techniques. As used herein, the term transformation encompasses all techniques by which a nucleic acid molecule might be introduced into such a cell, plant or animal cell, including transfection with viral vectors, transformation by Agrobacterium, with plasmid vectors, and introduction of naked DNA by electroporation, lipofection, and particle gun acceleration and includes transient as well as stable transformants.

The term “transgenic plant” refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

The term “vector” refers to a nucleic acid molecule which is used to introduce a polynucleotide sequence into a host cell, thereby producing a transformed host cell. A “vector” may comprise genetic material in addition to the above-described genetic construct, e.g., one or more nucleic acid sequences that permit it to replicate in one or more host cells, such as origin(s) of replication, selectable marker genes and other genetic elements known in the art (e.g., sequences for integrating the genetic material into the genome of the host cell, and so on).

### II. Positive Selection of Transgenic Plants

A selection system is provided that uses sorbitol dehydrogenase as a selectable marker and sorbitol as a selective agent for selecting genetically modified plants or plant cells. Positive selection methods have advantages over the more common negative selection methods. In negative selection methods, an introduced gene confers resistance to a toxic selective agent by detoxifying it. In contrast, positive selection introduces a gene which confers a growth advantage to the transformed cells, over the non-transformed cells. The data in the Examples demonstrate the ability of transformed cells expressing an enzyme having sorbitol dehydrogenase activity to proliferate in plant growth medium with sorbitol as the sole source of carbon, while untransformed plants remain dormant or slow growing. In a preferred embodiment biomass crops such as switchgrass are genetically engineered to express sorbitol dehydrogenase in an amount effective to allow the transformed switchgrass to use sorbitol as its sole source for carbon when grown in in tissue culture.

A. Sorbitol Dehydrogenase

Sorbitol dehydrogenase (EC 1.1.1.14) is an enzyme capable of converting sorbitol into fructose. Sorbitol dehydrogenase has been found primarily in rosaceous species (i.e., apples and peaches) in plants and also exists in bacteria. Since relatively few plant species can grow in the presence of sorbitol as a sole carbon source, expression of sorbitol dehydrogenase in transgenic plants and subsequent growth of the transformed plant material on sorbitol advantageously provides a positive selection method for many plant species.

The nucleic acid and protein sequences for sorbitol dehydrogenase from a variety of species are known in the art and can be used with the disclosed transgenic plants. For example, U.S. Pat. No. 6,544,756 to Uchida, et al. describes sorbitol dehydrogenase and microorganisms and processes for its production. U.S. Pat. Nos. 6,653,115 to Hoshino, et al. and 6,127,156 to Hoshino, et al. as well as U.S. Patent App. Pub. 2003/0022336 to Masuda, Ikuko, et al. describe genetic sequences encoding sorbitol dehydrogenase. U.S. Pat. No. 6,444,449 to Hoshino, et al. describes the use of sorbitol dehydrogenase and a sorbitol dehydrogenase gene in processes for producing L-sorbose via fermentation. None of the documents describe the use of sorbitol dehydrogenase as a selectable marker for plant transformation.

B. Vectors and Constructs

Vectors and constructs that express sorbitol dehydrogenase as a selectable marker and that allow for the selection of transgenic plants grown in the presence of sorbitol are also provided. The constructs can include an expression cassette containing the sorbitol dehydrogenase gene and one or more genes of interest encoding proteins, for example enzymes that can provide desired input or output traits to a plant. Transformation constructs can be engineered such that transformation of the nuclear genome and expression of transgenes from the nuclear genome occurs. Alternatively, transformation constructs can be engineered such that transformation of the plastid genome and expression from the plastid genome occurs. Preferred vectors and constructs are provided in the Examples, for example the nucleic acid sequence according to SEQ ID NO: 1, SEQ ID NO: 5 and SEQ ID NO: 6 or a complement thereof.

An exemplary construct contains operatively linked in the 5′ to 3′ direction, a promoter that directs transcription of a nucleic acid sequence, a nucleic acid sequence encoding a protein with sorbitol dehydrogenase activity, and a 3′ polyadenylation signal sequence. Typically, the encoded protein will have at least about 10, 20, 30, 40, 50, 60, 70, 80, 90, or 100 percent sorbitol dehydrogenase activity of sorbitol dehydrogenase from Pseudomonas sp. KS-E1806.

Generally, nucleic acid sequences encoding sorbitol dehydrogenase are first assembled in expression cassettes behind a suitable promoter expressible in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organdies and cell compartments. These expression cassettes can then be easily transferred to the plant transformation vectors. There are many plant transformation vector options available and representative plant transformation vectors are described in Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular biology—a laboratory course manual (1995), Maliga, P., Klessig, D. F., Cashmore, A. R., Gruissem, W. and Varner, J. E. eds. Cold Spring Laboratory Press, New York).

An additional approach is to use a vector to specifically transform the plant plastid chromosome by homologous recombination (U.S. Pat. No. 5,545,818 to McBride, et al.), in which case it is possible to take advantage of the prokaryotic nature of the plastid genome and insert a number of transgenes as an operon.

In a preferred embodiment, sorbitol dehydrogenase is used as a selectable marker in conjunction with the expression of transgenes that encode enzymes and other factors required for production of a biopolymer, such as a polyhydroxyalkanoate (PHA), a vegetable oil containing fatty acids with a desirable industrial or nutritional profile, a nutraceutical compound, plants with increased oil content, plants with increased cellulose content, plants with decreased lignin content, plants with increased drought tolerance, plants with increased water use efficiency and plants with increased nitrogen use efficiency.

The following is a description of various components of typical expression cassettes.

1. Promoters

The selection of the promoter used in expression cassettes determine the spatial and temporal expression pattern of the transgene in the transgenic plant. Selected promoters express transgenes in specific cell types (such as leaf epidermal cells, mesophyll cells, root cortex cells) or in specific tissues or organs (roots, leaves or flowers, for example) and the selection reflects the desired location of accumulation of the gene product. Alternatively, the selected promoter drives expression of the gene under various inducing conditions.

Promoters vary in their strength, i.e., ability to promote transcription. Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044 to Ryals, et al.).

A suitable category of promoters is that which is wound inducible. Numerous promoters have been described which are expressed at wound sites. Preferred promoters of this kind include those described by Stanford et al. Mol. Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Suitable tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth & Grula, Plant Molec. Biol. 12: 579-589 (1989)). A suitable promoter for root specific expression is that described by de Framond FEBS 290: 103-106 (1991); EP 0 452 269 to de Framond and a root-specific promoter is that from the T-1 gene. A suitable stem specific promoter is that described in U.S. Pat. No. 5,625,136 and which drives expression of the maize trpA gene.

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

At the extreme 3′ end of the transcript, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, M., et al., Nucleic Acids Res., 11, 369-385 (1983)).

3. Sequences for the Enhancement or Regulation of Expression

Numerous sequences have been found to enhance gene expression from within the transcriptional unit and these sequences can be used in conjunction with the genes to increase their expression in transgenic plants. For example, various intron sequences such as introns of the maize Adh1 gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene may be genetically engineered by altering the coding sequence for increased or optimal expression in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al, Biotechnol. 11: 194 (1993)).

5. Targeting Sequences

The disclosed vectors and constructs may further include, within the region that encodes the protein to be expressed, one or more nucleotide sequences encoding a targeting sequence. A “targeting” sequence is a nucleotide sequence that encodes an amino acid sequence or motif that directs the encoded protein to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The “targeting” sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof, such as a glyoxysome) an endoplasmic reticulum, an endosome, a vacuole, a plasma membrane, a cell wall, a mitochondria, a chloroplast or a plastid. A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, A. & Trelease, R. N. Plant Physiol., 107:1201-1208 (1995); T. P. Wallace et al., “Plant Organellular Targeting Sequences,” in Plant Molecular Biology, Ed. R. Croy, BIOS Scientific Publishers Limited (1993) pp. 287-288, and peroxisomal targeting in plant is shown in M. Volokita, The Plant J., 361-366 (1991)).

C. Plants and Tissues for Transfection

Both dicotyledons and monocotyledons can be used in the disclosed positive selection system. Representative plants useful in the methods disclosed herein include the Brassica family including napus, rappa, sp. carinata and juncea; industrial oilseeds such as Camelina sativa, Crambe, Jatropha, castor; Arabidopsis thaliana; soybean; cottonseed; sunflower; palm; coconut; rice; safflower; peanut; mustards including Sinapis alba; sugarcane and flax. Crops harvested as biomass, such as silage corn, alfalfa, switchgrass, miscanthus, sorghum or tobacco, also are useful with the methods disclosed herein. Representative tissues for transformation using these vectors include protoplasts, cells, callus tissue, leaf discs, pollen, and meristems. Algae can also be used. Representative species of algae include, but are not limited to Emiliana Huxleyi; Arthrospira platensis (Spirolina); Haematococcus pluvialis; Dunaliella salina; and Chlamydomonas reinhardii.

D. Transgenes

Genes that alter the metabolism of plants can be used with the disclosed positive selection system. The expression of multiple enzymes is useful for altering the metabolism of plants to increase, for example, the levels of nutritional amino acids (Falco et al. Biotechnology 13: 577 (1995)), to modify lignin metabolism (Baucher et al. Crit. Rev. Biochem. Mol. 38: 305-350 (2003)), to modify oil compositions (Drexler et al. J. Plant Physiol. 160: 779-802 (2003)), to modify starch, or to produce polyhydroxyalkanoate polymers (Huisman and Madison, Microbial and Mol. Biol. Rev. 63: 21-53 (1999). In preferred embodiments, the product of the transgenes is a biopolymer, such as a polyhydroxyalkanoate (PHA), a vegetable oil containing fatty acids with a desirable industrial or nutritional profile, or a nutraceutical compound.

### III. Methods of Making Transgenic Plants

A. Plant Transformation Techniques

The transformation of suitable agronomic plant hosts using vectors expressing sorbitol dehydrogenase can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al.; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga, et al. eds.) Cold Spring Laboratory Press, New York (1995)).

Soybean can be transformed by a number of reported procedures (U.S. Pat. Nos. 5,015,580 to Christou, et al.; 5,015,944 to Bubash; 5,024,944 to Collins, et al.; 5,322,783 to Tomes, et al.; 5,416,011 to Hinchee, et al.; 5,169,770 to Chee, et al.).

A number of transformation procedures have been reported for the production of transgenic maize plants including pollen transformation (U.S. Pat. No. 5,629,183 to Saunders, et al.), silicon fiber-mediated transformation (U.S. Pat. No. 5,464,765 to Coffee, et al.), electroporation of protoplasts (U.S. Pat. Nos. 5,231,019 Paszkowski, et al.; 5,472,869 to Krzyzek, et al.; 5,384,253 to Krzyzek, et al.), gene gun (U.S. Pat. Nos. 5,538,877 to Lundquist, et al. and 5,538,880 to Lundquist, et al.), and Agrobacterium-mediated transformation (EP 0 604 662 A1 and WO 94/00977 both to Hiei Yukou et al.). The Agrobacterium-mediated procedure is particularly preferred as single integration events of the transgene constructs are more readily obtained using this procedure which greatly facilitates subsequent plant breeding. Cotton can be transformed by particle bombardment (U.S. Pat. Nos. 5,004,863 to Umbeck and 5,159,135 to Umbeck). Sunflower can be transformed using a combination of particle bombardment and Agrobacterium infection (EP 0 486 233 A2 to Bidney, Dennis; U.S. Pat. No. 5,030,572 to Power, et al.). Flax can be transformed by either particle bombardment or Agrobacterium-mediated transformation. Switchgrass can be transformed using either biolistic or Agrobacterium mediated methods (Richards et al. Plant Cell Rep. 20: 48-54 (2001); Somleva et al. Crop Science 42: 2080-2087 (2002)). Methods for sugarcane transformation have also been described (Franks & Birch Aust. J. Plant Physiol. 18, 471-480 (1991); WO 2002/037951 to Elliott, Adrian, Ross, et al).

Recombinase technologies which are useful in practicing the current invention include the cre-lox, FLP/FRT and Gin systems. Methods by which these technologies can be used for the purpose described herein are described for example in (U.S. Pat. No. 5,527,695 to Hodges, et al.; Dale And Ow, Proc. Natl. Acad. Sci. USA, 88:10558-10562 (1991); Medberry et al., Nucleic Acids Res., 23: 485-490 (1995)).

Engineered minichromosomes can also be used to express one or more genes in plant cells. Cloned telomeric repeats introduced into cells may truncate the distal portion of a chromosome by the formation of a new telomere at the integration site. Using this method, a vector for gene transfer can be prepared by trimming off the arms of a natural plant chromosome and adding an insertion site for large inserts (Yu et al., Proc Natl Acad Sci USA, 2006, 103, 17331-6; Yu et al., Proc Natl Acad Sci USA, 2007, 104, 8924-9). The utility of engineered minichromosome platforms has been shown using Cre/lox and FRT/FLP site-specific recombination systems on a maize minichromosome where the ability to undergo recombination was demonstrated (Yu et al., Proc Natl Acad Sci USA, 2006, 103, 17331-6; Yu et al., Proc Natl Acad Sci USA, 2007, 104, 8924-9). Such technologies could be applied to minichromosomes, for example, to add genes to an engineered plant. Site specific recombination systems have also been demonstrated to be valuable tools for marker gene removal (Kerbach, S. et al., Theor Appl Genet, 2005, 111, 1608-1616), gene targeting (Chawla, R et al., Plant Biotechnol J, 2006, 4, 209-218; Choi, S. et al., Nucleic Acids Res, 2000, 28, E19; Srivastava, V, & Ow, D W, Plant Mol Biol, 2001, 46, 561-566; Lyznik, L A, et al., Nucleic Acids Res, 1993, 21, 969-975), and gene conversion (Djukanovic, V, et al., Plant Biotechnol J, (2006, 4, 345-357).

An alternative approach to chromosome engineering in plants involves in vivo assembly of autonomous plant minichromosomes (Carlson et al., PLoS Genet, 2007, 3, 1965-74). Plant cells can be transformed with centromeric sequences and screened for plants that have assembled autonomous chromosomes de novo. Useful constructs combine a selectable marker gene with genomic DNA fragments containing centromeric satellite and retroelement sequences and/or other repeats.

Another approach useful to the described invention is Engineered Trait Loci (“ETL”) technology (U.S. Pat. No. 6,077,697; US Patent Application 2006/0143732). This system targets DNA to a heterochromatic region of plant chromosomes, such as the pericentric heterochromatin, in the short arm of acrocentric chromosomes. Targeting sequences may include ribosomal DNA (rDNA) or lambda phage DNA. The pericentric rDNA region supports stable insertion, low recombination, and high levels of gene expression. This technology is also useful for stacking of multiple traits in a plant (US Patent Application 2006/0246586).

Zinc-finger nucleases (ZFNs) are also useful for practicing the invention in that they allow double strand DNA cleavage at specific sites in plant chromosomes such that targeted gene insertion or deletion can be performed (Shukla et al., Nature, 2009; Townsend et al., Nature, 2009).

Following transformation by any one of the methods described above, the following procedures can, for example, be used to obtain a transformed plant expressing the transgenes: select the plant cells that have been transformed on a selective medium, in particular sorbitol as the sole carbon source; regenerate the plant cells that have been transformed to produce differentiated plants; select transformed plants expressing the transgene producing the desired level of desired polypeptide(s) in the desired tissue and cellular location.

Transformation techniques for dicotyledons are well known in the art and include Agrobacterium-based techniques and techniques that do not require Agrobacterium. Non Agrobacterium techniques involve the uptake of exogenous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art.

B. Plastid Transformation

Another embodiment provides a transgene(s), for example sorbitol dehydrogenase and one or more additional transgenes of interest, directly transformed into the plastid genome. Plastid transformation technology is extensively described in U.S. Pat. Nos. 5,451,513 to Maliga, et al., 5,545,817 to McBride, et al., and 5,545,818 to McBride, et al., in PCT application no. WO 95/16783 to McBride et al., and in McBride et al. Proc. Natl. Acad. Sci. USA 91:7301-7305 (1994). The basic technique for chloroplast transformation involves introducing regions of cloned plastid DNA flanking a selectable marker together with the gene(s) of interest into a suitable target tissue, e.g., using biolistics or protoplast transformation (e.g., calcium chloride or PEG mediated transformation). The 1 to 1.5 kb flanking regions facilitate homologous recombination with the plastid genome and thus allow the replacement or modification of specific regions of the plastome. Suitable plastids that can be transfected include, but are not limited to chloroplasts, etioplasts, chromoplasts, leucoplasts, amyloplasts, statoliths, elaioplasts, proteinoplasts and combinations thereof.

### EXAMPLES

### Example 1

### Growth of Switchgrass Callus Cultures in the Presence of Different Carbon Sources

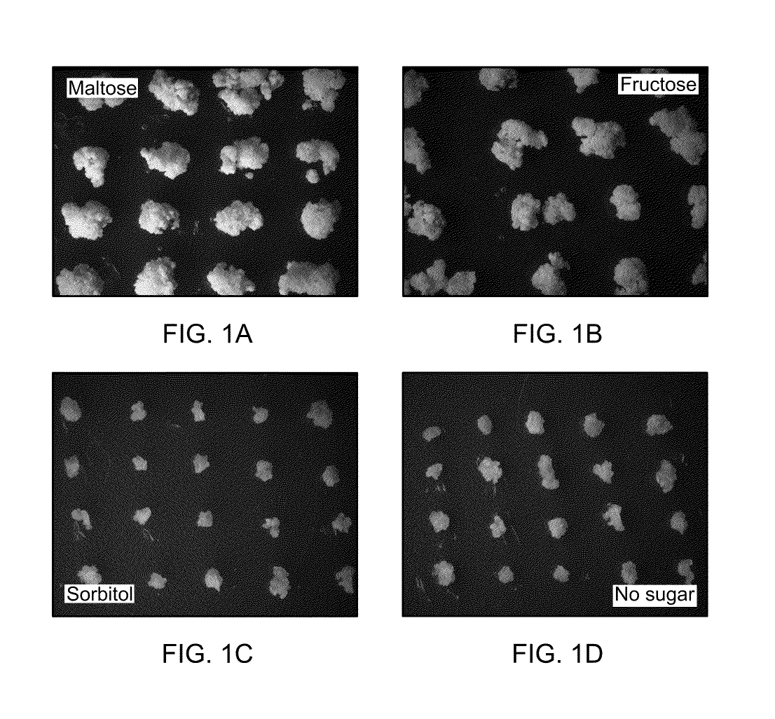

The in vitro response of various plants grown on medium supplemented with different sugar sources was investigated. For these purposes, switchgrass (Panicum virgatum L. cv. ‘Alamo’) was chosen as a representative monocot species. Highly embryogenic callus cultures of switchgrass were initiated from mature caryopses according to established procedures (Denchev, P. D. and B. V. Conger, Crop Sci., 34: 1623-1627 (1994)) and transferred to callus multiplication media [media consists of MS basal salts (product#MS002, Caisson Laboratories, North Logan, Utah, USA), 6-benzylaminopurine (BAP, 4.4 mM), 2,4-dichlorophenoxyacetic acid (2,4-D, 22.6 mM), and agar (8 g/L agar), pH 5.6]. The media was supplemented with carbon sources as indicated in the following concentrations: maltose (83.3 mM), fructose (111 mM), sorbitol (41.2 mM), or no carbon source. After 4 weeks of dark incubation at 28° C., the callus multiplication ability in the presence of various carbon supplements or no carbon supplement was visually examined. Cultures of switchgrass incubated on medium containing maltose or fructose were able to proliferate normally and displayed considerable callus growth (FIG. 1). In contrast, cultures incubated on medium containing sorbitol and medium without a carbon source remained dormant with minimal or no incremental growth (FIG. 1). These experiments indicated that sorbitol could not be used as a sole carbon source for growth of switchgrass cultures. These experiments further suggested that expression of a gene encoding an enzyme that could convert sorbitol to fructose, such as sdh, might enable the growth of cultures on a medium that contained sorbitol as a sole carbon source.

### Example 2

### Evaluation of Calli Growth with In Vitro Cultures of Arabidopsis thaliana in the Presence of Different Carbon Sources

Growth of cultures of Arabidopsis thaliana, a model dicot species, were also examined to determine if they were able to grow in the presence of sorbitol as a sole carbon source. Leaf and root explants were excised from sterile seedlings of Arabidopsis and were plated on medium containing maltose, fructose, or sorbitol, or no carbon supplement as described in Example 1. After 4 weeks of dark incubation at 25° C., both root and leaf cultures showed considerable callus growth in the presence of maltose and fructose. As with switchgrass callus cultures, little to no growth of Arabidopsis cultures derived from leaves or roots was observed on medium containing sorbitol or on medium without a carbon source.

### Example 3

### Construction of Plasmid for Expression of Sorbitol Dehydrogenase

To determine whether expression of sdh, a gene encoding sorbitol dehydrogenase that catalyzes the conversion of sorbitol to fructose, could enable cultures of switchgrass to grow in the presence of sorbitol, a plant transformation construct for Agrobacterium-mediated transformation of switchgrass was designed and constructed. Genes encoding sorbitol dehydrogenase have been cloned from many organisms including Bacillus subtilis (Ng, K., et al., J. Biol. Chem., 267(35): 24989-24994 (1992); Gluconobacter suboxydans (U.S. Pat. No. 6,127,156 to Hoshino, et al.), Homo sapiens (Lee, F. K., et al. Genomics, 21(2): 354-358 (1994), apple fruit (Yamada, K., et al., Plant Cell Physiol. 39(12): 1375-1379 (1998), Saccharomyces cerevisiae (Sarthy, A., et al., Gene, 140(1): 121-126 (1994), and Pseudomonas sp. KS-E1806 (EP1262551 to Masuda, Ikuko, et al.). For the purposes of this study, the sorbitol dehydrogenase gene from Pseudomonas sp. KS-E1806 was used.

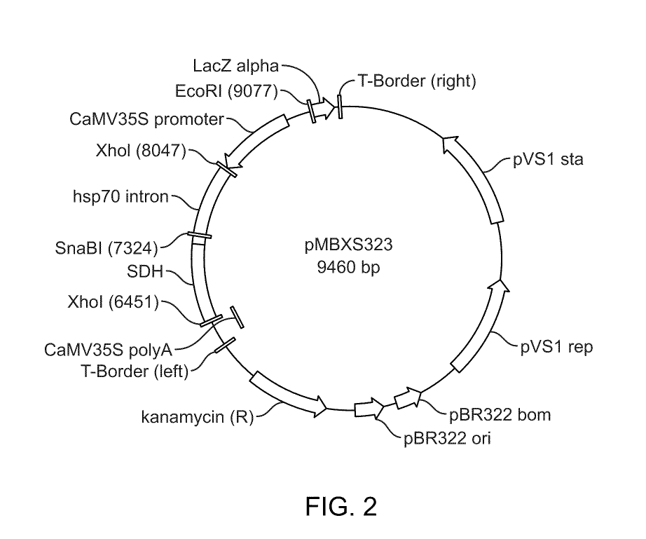

Plasmid pMBXS323 (FIG. 2) is a derivative of plant transformation construct pCAMBIA3300 (Center for Application of Molecular Biology to International Agriculture, Canberra, Australia) and contains the CaMV35S promoter (Kay, R., et al., Science, 236: 1299-1302 (1987)), the hsp70 intron (U.S. Pat. No. 5,593,874 to Brown, et al.) for enhanced expression in monocots, the sorbitol dehydrogenase gene (sdh) from Pseudomonas sp. KS-E1806, and the CaMV35S polyadenylation sequence Odell, J., et al., Nature, 313(6005): 810-812 (1985)).

The nucleotide sequence of plasmid pMBXS323 is as follows.

| (SEQ ID NO: 1) | |
|---|---|
| 1 | CATGCCAACC ACAGGGTTCC CCTCGGGATC AAAGTACTTT GATCCAACCC |
| 51 | CTCCGCTGCT ATAGTGCAGT CGGCTTCTGA CGTTCAGTGC AGCCGTCTTC |
| 101 | TGAAAACGAC ATGTCGCACA AGTCCTAAGT TACGCGACAG GCTGCCGCCC |
| 151 | TGCCCTTTTC CTGGCGTTTT CTTGTCGCGT GTTTTAGTCG CATAAAGTAG |
| 201 | AATACTTGCG ACTAGAACCG GAGACATTAC GCCATGAACA AGAGCGCCGC |
| 251 | CGCTGGCCTG CTGGGCTATG CCCGCGTCAG CACCGACGAC CAGGACTTGA |
| 301 | CCAACCAACG GGCCGAACTG CACGCGGCCG GCTGCACCAA GCTGTTTTCC |
| 351 | GAGAAGATCA CCGGCACCAG GCGCGACCGC CCGGAGCTGG CCAGGATGCT |
| 401 | TGACCACCTA CGCCCTGGCG ACGTTGTGAC AGTGACCAGG CTAGACCGCC |
| 451 | TGGCCCGCAG CACCCGCGAC CTACTGGACA TTGCCGAGCG CATCCAGGAG |
| 501 | GCCGGCGCGG GCCTGCGTAG CCTGGCAGAG CCGTGGGCCG ACACCACCAC |
| 551 | GCCGGCCGGC CGCATGGTGT TGACCGTGTT CGCCGGCATT GCCGAGTTCG |
| 601 | AGCGTTCCCT AATCATCGAC CGCACCCGGA GCGGGCGCGA GGCCGCCAAG |
| 651 | GCCCGAGGCG TGAAGTTTGG CCCCCGCCCT ACCCTCACCC CGGCACAGAT |
| 701 | CGCGCACGCC CGCGAGCTGA TCGACCAGGA AGGCCGCACC GTGAAAGAGG |
| 751 | CGGCTGCACT GCTTGGCGTG CATCGCTCGA CCCTGTACCG CGCACTTGAG |
| 801 | CGCAGCGAGG AAGTGACGCC CACCGAGGCC AGGCGGCGCG GTGCCTTCCG |
| 851 | TGAGGACGCA TTGACCGAGG CCGACGCCCT GGCGGCCGCC GAGAATGAAC |
| 901 | GCCAAGAGGA ACAAGCATGA AACCGCACCA GGACGGCCAG GACGAACCGT |
| 951 | TTTTCATTAC CGAAGAGATC GAGGCGGAGA TGATCGCGGC CGGGTACGTG |
| 1001 | TTCGAGCCGC CCACGCACGT CTCAACCGTG CGGCTGCATG AAATCCTGGC |
| 1051 | CGGTTTGTCT GATGCCAAGC TGGCGGCCTG GCCGGCCAGC TTGGCCGCTG |
| 1101 | AAGAAACCGA GCGCCGCCGT CTAAAAAGGT GATGTGTATT TGAGTAAAAC |
| 1151 | AGCTTGCGTC ATGCGGTCGC TGCGTATATG ATGCGATGAG TAAATAAACA |
| 1201 | AATACGCAAG GGGAACGCAT GAAGGTTATC GCTGTACTTA ACCAGAAAGG |
| 1251 | CGGGTCAGGC AAGACGACCA TCGCAACCCA TCTAGCCCGC GCCCTGCAAC |
| 1301 | TCGCCGGGGC CGATGTTCTG TTAGTCGATT CCGATCCCCA GGGCAGTGCC |
| 1351 | CGCGATTGGG CGGCCGTGCG GGAAGATCAA CCGCTAACCG TTGTCGGCAT |
| 1401 | CGACCGCCCG ACGATTGACC GCGACGTGAA GGCCATCGGC CGGCGCGACT |
| 1451 | TCGTAGTGAT CGACGGAGCG CCCCAGGCGG CGGACTTGGC TGTGTCCGCG |
| 1501 | ATCAAGGCAG CCGACTTCGT GCTGATTCCG GTGCAGCCAA GCCCTTACGA |
| 1551 | CATATGGGCC ACCGCCGACC TGGTGGAGCT GGTTAAGCAG CGCATTGAGG |
| 1601 | TCACGGATGG AAGGCTACAA GCGGCCTTTG TCGTGTCGCG GGCGATCAAA |
| 1651 | GGCACGCGCA TCGGCGGTGA GGTTGCCGAG GCGCTGGCCG GGTACGAGCT |
| 1701 | GCCCATTCTT GAGTCCCGTA TCACGCAGCG CGTGAGCTAC CCAGGCACTG |
| 1751 | CCGCCGCCGG CACAACCGTT CTTGAATCAG AACCCGAGGG CGACGCTGCC |
| 1801 | CGCGAGGTCC AGGCGCTGGC CGCTGAAATT AAATCAAAAC TCATTTGAGT |
| 1851 | TAATGAGGTA AAGAGAAAAT GAGCAAAAGC ACAAACACGC TAAGTGCCGG |
| 1901 | CCGTCCGAGC GCACGCAGCA GCAAGGCTGC AACGTTGGCC AGCCTGGCAG |
| 1951 | ACACGCCAGC CATGAAGCGG GTCAACTTTC AGTTGCCGGC GGAGGATCAC |
| 2001 | ACCAAGCTGA AGATGTACGC GGTACGCCAA GGCAAGACCA TTACCGAGCT |
| 2051 | GCTATCTGAA TACATCGCGC AGCTACCAGA GTAAATGAGC AAATGAATAA |
| 2101 | ATGAGTAGAT GAATTTTAGC GGCTAAAGGA GGCGGCATGG AAAATCAAGA |
| 2151 | ACAACCAGGC ACCGACGCCG TGGAATGCCC CATGTGTGGA GGAACGGGCG |
| 2201 | GTTGGCCAGG CGTAAGCGGC TGGGTTGTCT GCCGGCCCTG CAATGGCACT |
| 2251 | GGAACCCCCA AGCCCGAGGA ATCGGCGTGA CGGTCGCAAA CCATCCGGCC |
| 2301 | CGGTACAAAT CGGCGCGGCG CTGGGTGATG ACCTGGTGGA GAAGTTGAAG |
| 2351 | GCCGCGCAGG CCGCCCAGCG GCAACGCATC GAGGCAGAAG CACGCCCCGG |
| 2401 | TGAATCGTGG CAAGCGGCCG CTGATCGAAT CCGCAAAGAA TCCCGGCAAC |
| 2451 | CGCCGGCAGC CGGTGCGCCG TCGATTAGGA AGCCGCCCAA GGGCGACGAG |
| 2501 | CAACCAGATT TTTTCGTTCC GATGCTCTAT GACGTGGGCA CCCGCGATAG |
| 2551 | TCGCAGCATC ATGGACGTGG CCGTTTTCCG TCTGTCGAAG CGTGACCGAC |
| 2601 | GAGCTGGCGA GGTGATCCGC TACGAGCTTC CAGACGGGCA CGTAGAGGTT |
| 2651 | TCCGCAGGGC CGGCCGGCAT GGCCAGTGTG TGGGATTACG ACCTGGTACT |
| 2701 | GATGGCGGTT TCCCATCTAA CCGAATCCAT GAACCGATAC CGGGAAGGGA |
| 2751 | AGGGAGACAA GCCCGGCCGC GTGTTCCGTC CACACGTTGC GGACGTACTC |
| 2801 | AAGTTCTGCC GGCGAGCCGA TGGCGGAAAG CAGAAAGACG ACCTGGTAGA |
| 2851 | AACCTGCATT CGGTTAAACA CCACGCACGT TGCCATGCAG CGTACGAAGA |
| 2901 | AGGCCAAGAA CGGCCGCCTG GTGACGGTAT CCGAGGGTGA AGCCTTGATT |
| 2951 | AGCCGCTACA AGATCGTAAA GAGCGAAACC GGGCGGCCGG AGTACATCGA |
| 3001 | GATCGAGCTA GCTGATTGGA TGTACCGCGA GATCACAGAA GGCAAGAACC |
| 3051 | CGGACGTGCT GACGGTTCAC CCCGATTACT TTTTGATCGA TCCCGGCATC |
| 3101 | GGCCGTTTTC TCTACCGCCT GGCACGCCGC GCCGCAGGCA AGGCAGAAGC |
| 3151 | CAGATGGTTG TTCAAGACGA TCTACGAACG CAGTGGCAGC GCCGGAGAGT |
| 3201 | TCAAGAAGTT CTGTTTCACC GTGCGCAAGC TGATCGGGTC AAATGACCTG |
| 3251 | CCGGAGTACG ATTTGAAGGA GGAGGCGGGG CAGGCTGGCC CGATCCTAGT |
| 3301 | CATGCGCTAC CGCAACCTGA TCGAGGGCGA AGCATCCGCC GGTTCCTAAT |
| 3351 | GTACGGAGCA GATGCTAGGG CAAATTGCCC TAGCAGGGGA AAAAGGTCGA |
| 3401 | AAAGGTCTCT TTCCTGTGGA TAGCACGTAC ATTGGGAACC CAAAGCCGTA |
| 3451 | CATTGGGAAC CGGAACCCGT ACATTGGGAA CCCAAAGCCG TACATTGGGA |
| 3501 | ACCGGTCACA CATGTAAGTG ACTGATATAA AAGAGAAAAA AGGCGATTTT |
| 3551 | TCCGCCTAAA ACTCTTTAAA ACTTATTAAA ACTCTTAAAA CCCGCCTGGC |
| 3601 | CTGTGCATAA CTGTCTGGCC AGCGCACAGC CGAAGAGCTG CAAAAAGCGC |
| 3651 | CTACCCTTCG GTCGCTGCGC TCCCTACGCC CCGCCGCTTC GCGTCGGCCT |
| 3701 | ATCGCGGCCG CTGGCCGCTC AAAAATGGCT GGCCTACGGC CAGGCAATCT |
| 3751 | ACCAGGGCGC GGACAAGCCG CGCCGTCGCC ACTCGACCGC CGGCGCCCAC |
| 3801 | ATCAAGGCAC CCTGCCTCGC GCGTTTCGGT GATGACGGTG AAAACCTCTG |
| 3851 | ACACATGCAG CTCCCGGAGA CGGTCACAGC TTGTCTGTAA GCGGATGCCG |
| 3901 | GGAGCAGACA AGCCCGTCAG GGCGCGTCAG CGGGTGTTGG CGGGTGTCGG |
| 3951 | GGCGCAGCCA TGACCCAGTC ACGTAGCGAT AGCGGAGTGT ATACTGGCTT |
| 4001 | AACTATGCGG CATCAGAGCA GATTGTACTG AGAGTGCACC ATATGCGGTG |
| 4051 | TGAAATACCG CACAGATGCG TAAGGAGAAA ATACCGCATC AGGCGCTCTT |
| 4101 | CCGCTTCCTC GCTCACTGAC TCGCTGCGCT CGGTCGTTCG GCTGCGGCGA |
| 4151 | GCGGTATCAG CTCACTCAAA GGCGGTAATA CGGTTATCCA CAGAATCAGG |
| 4201 | GGATAACGCA GGAAAGAACA TGTGAGCAAA AGGCCAGCAA AAGGCCAGGA |
| 4251 | ACCGTAAAAA GGCCGCGTTG CTGGCGTTTT TCCATAGGCT CCGCCCCCCT |
| 4301 | GACGAGCATC ACAAAAATCG ACGCTCAAGT CAGAGGTGGC GAAACCCGAC |
| 4351 | AGGACTATAA AGATACCAGG CGTTTCCCCC TGGAAGCTCC CTCGTGCGCT |
| 4401 | CTCCTGTTCC GACCCTGCCG CTTACCGGAT ACCTGTCCGC CTTTCTCCCT |
| 4451 | TCGGGAAGCG TGGCGCTTTC TCATAGCTCA CGCTGTAGGT ATCTCAGTTC |
| 4501 | GGTGTAGGTC GTTCGCTCCA AGCTGGGCTG TGTGCACGAA CCCCCCGTTC |
| 4551 | AGCCCGACCG CTGCGCCTTA TCCGGTAACT ATCGTCTTGA GTCCAACCCG |
| 4601 | GTAAGACACG ACTTATCGCC ACTGGCAGCA GCCACTGGTA ACAGGATTAG |
| 4651 | CAGAGCGAGG TATGTAGGCG GTGCTACAGA GTTCTTGAAG TGGTGGCCTA |
| 4701 | ACTACGGCTA CACTAGAAGG ACAGTATTTG GTATCTGCGC TCTGCTGAAG |
| 4751 | CCAGTTACCT TCGGAAAAAG AGTTGGTAGC TCTTGATCCG GCAAACAAAC |
| 4801 | CACCGCTGGT AGCGGTGGTT TTTTTGTTTG CAAGCAGCAG ATTACGCGCA |
| 4851 | GAAAAAAAGG ATCTCAAGAA GATCCTTTGA TCTTTTCTAC GGGGTCTGAC |
| 4901 | GCTCAGTGGA ACGAAAACTC ACGTTAAGGG ATTTTGGTCA TGCATTCTAG |
| 4951 | GTACTAAAAC AATTCATCCA GTAAAATATA ATATTTTATT TTCTCCCAAT |
| 5001 | CAGGCTTGAT CCCCAGTAAG TCAAAAAATA GCTCGACATA CTGTTCTTCC |
| 5051 | CCGATATCCT CCCTGATCGA CCGGACGCAG AAGGCAATGT CATACCACTT |
| 5101 | GTCCGCCCTG CCGCTTCTCC CAAGATCAAT AAAGCCACTT ACTTTGCCAT |
| 5151 | CTTTCACAAA GATGTTGCTG TCTCCCAGGT CGCCGTGGGA AAAGACAAGT |
| 5201 | TCCTCTTCGG GCTTTTCCGT CTTTAAAAAA TCATACAGCT CGCGCGGATC |
| 5251 | TTTAAATGGA GTGTCTTCTT CCCAGTTTTC GCAATCCACA TCGGCCAGAT |
| 5301 | CGTTATTCAG TAAGTAATCC AATTCGGCTA AGCGGCTGTC TAAGCTATTC |
| 5351 | GTATAGGGAC AATCCGATAT GTCGATGGAG TGAAAGAGCC TGATGCACTC |
| 5401 | CGCATACAGC TCGATAATCT TTTCAGGGCT TTGTTCATCT TCATACTCTT |
| 5451 | CCGAGCAAAG GACGCCATCG GCCTCACTCA TGAGCAGATT GCTCCAGCCA |
| 5501 | TCATGCCGTT CAAAGTGCAG GACCTTTGGA ACAGGCAGCT TTCCTTCCAG |
| 5551 | CCATAGCATC ATGTCCTTTT CCCGTTCCAC ATCATAGGTG GTCCCTTTAT |
| 5601 | ACCGGCTGTC CGTCATTTTT AAATATAGGT TTTCATTTTC TCCCACCAGC |
| 5651 | TTATATACCT TAGCAGGAGA CATTCCTTCC GTATCTTTTA CGCAGCGGTA |
| 5701 | TTTTTCGATC AGTTTTTTCA ATTCCGGTGA TATTCTCATT TTAGCCATTT |
| 5751 | ATTATTTCCT TCCTCTTTTC TACAGTATTT AAAGATACCC CAAGAAGCTA |
| 5801 | ATTATAACAA GACGAACTCC AATTCACTGT TCCTTGCATT CTAAAACCTT |
| 5851 | AAATACCAGA AAACAGCTTT TTCAAAGTTG TTTTCAAAGT TGGCGTATAA |
| 5901 | CATAGTATCG ACGGAGCCGA TTTTGAAACC GCGGTGATCA CAGGCAGCAA |
| 5951 | CGCTCTGTCA TCGTTACAAT CAACATGCTA CCCTCCGCGA GATCATCCGT |
| 6001 | GTTTCAAACC CGGCAGCTTA GTTGCCGTTC TTCCGAATAG CATCGGTAAC |
| 6051 | ATGAGCAAAG TCTGCCGCCT TACAACGGCT CTCCCGCTGA CGCCGTCCCG |
| 6101 | GACTGATGGG CTGCCTGTAT CGAGTGGTGA TTTTGTGCCG AGCTGCCGGT |
| 6151 | CGGGGAGCTG TTGGCTGGCT GGTGGCAGGA TATATTGTGG TGTAAACAAA |
| 6201 | TTGACGCTTA GACAACTTAA TAACACATTG CGGACGTTTT TAATGTACTG |
| 6251 | AATTAACGCC GAATTAATTC GGGGGATCTG GATTTTAGTA CTGGATTTTG |
| 6301 | GTTTTAGGAA TTAGAAATTT TATTGATAGA AGTATTTTAC AAATACAAAT |
| 6351 | ACATACTAAG GGTTTCTTAT ATGCTCAACA CATGAGCGAA ACCCTATAGG |
| 6401 | AACCCTAATT CCCTTATCTG GGAACTACTC ACACATTATT ATGGAGAAAC |
| 6451 | TCGAGTCAGC TCATCCAGTT GACGCCATCG ACGTTAAGGG TCTGGGCGGT |
| 6501 | GATGTAGTCG GCATCGGCCG ACGCGAGGAA CAGCGCGGCG CCCGTCAGGT |
| 6551 | CGCCCGGCAC GCCCATGCGG CCGAGCGGCA CGGCTTCACC GACGAGCCGC |
| 6601 | TTCTTCTCGC CGAGCGGCCG GTTCTCGTAG CGCGCGAACA GCGCATCGAC |
| 6651 | CTGCTCCCAC ATCGGCGTGT CGACCACGCC CGGCGCGATG CCGTTCACGT |
| 6701 | TGATCCGGTG CGGCGCGAGC GCGAGCGCGG CCGACTGCGT ATAGCTGATC |
| 6751 | ACCGCGGCCT TGGTCGCGCA GTAGTGCGAA ACGAGCGCCT CGCCGCGACG |
| 6801 | GCCGGCCTGC GACGACATGT TGACGATCTT GCCGCCGCGC CCCTGCTCGA |
| 6851 | CCATCCGTTG CGCAACCGCC TGCATCAGGA AGAACAGCCC TTTCACGTTG |
| 6901 | ACCGAGAACA GCCGGTCGAA CACGTCCCAG GATTCATCGA GGAGCGGACG |
| 6951 | CATGTCGAAC AGCGCCGCGT TGTTGAACAG AATGTCGACG CCGCCGAAGC |
| 7001 | GCTCGACCGC CGTGGCGACG ATCCGCGTGA TGTCGTCGCG ACGCGTGACG |
| 7051 | TCGGCCGTGA CGGCCACCGC GCGGCCCGGG TTGGCCTCGA TCAGCCGCGC |
| 7101 | GAGCGAGCCG CCTGCCGGCT TCACGTCGAC GAGCACGCAG CGCGCGCCCT |
| 7151 | CGTCCAGATA GCGTTGTGCG ACCGCCTCGC CGATGCCGCT TGCGGCGCCC |
| 7201 | GTCAGGATCG CGACCTTGTC TTCCAGTCTC ATTTTGCCGC TTGGTATCTG |
| 7251 | CATTACAATG AAATGAGCAA AGACTATGTG AGTAACACTG GTCAACACTA |
| 7301 | GGAGAAGGCA TCGAGCAAGA TACGTATGTA AAGAGAAGCA ATATAGTATC |
| 7351 | AGTTGGTAGA TACTAGATAC CATCAGGAGG TAAGGAGAGC AACAAAAAGG |
| 7401 | AAACTCTTTA TTTTTAAATT TTGTTACAAC AAACAAGCAG ATCAATGCAT |
| 7451 | CAAAATACTG TCAGTACTTA TTTCTTCAGA CAACAATATT TAAAACAAGT |
| 7501 | GCATCTGATC TTGACTTATG GTCACAATAA AGGAGCAGAG ATAAACATCA |
| 7551 | AAATTTCGTC ATTTATATTT ATTCCTTCAG GCGTTAACAA TTTAACAGCA |
| 7601 | CACAAACAAA AACAGAATAG GAATATCTAA TTTTGGCAAA TAATAAGCTC |
| 7651 | TGCAGACGAA CAAATTATTA TAGTATCGCC TATAATATGA ATCCCTATAC |
| 7701 | TATTGACCCA TATAATATGA AGCCTGTGCC TAAATTAACA GCAAACTTCT |
| 7751 | GAATCCAAGT GCCCTATAAC ACCAACATGT GCTTAAATAA ATACCGCTAA |
| 7801 | GCACCAAATT ACACATTTCT CGTATTGCTG TGTAGGTTCT ATCTTCGTTT |
| 7851 | CGTACTACCA TGTCCCTATA TTTTGCTGCT ACAAAGGACG GCAAGTAATC |
| 7901 | AGCACAGGCA GAACACGATT TCAGAGTGTA ATTCTAGATC CAGCTAAACC |
| 7951 | ACTCTCAGCA ATCACCACAC AAGAGAGCAT TCAGAGAAAC GTGGCAGTAA |
| 8001 | CAAAGGCAGA GGGCGGAGTG AGCGCGTACC GAAGACGGTA GATCTCTCGA |
| 8051 | GAGAGATAGA TTTGTAGAGA GAGACTGGTG ATTTCAGCGT GTCCTCTCCA |
| 8101 | AATGAAATGA ACTTCCTTAT ATAGAGGAAG GTCTTGCGAA GGATAGTGGG |
| 8151 | ATTGTGCGTC ATCCCTTACG TCAGTGGAGA TATCACATCA ATCCACTTGC |
| 8201 | TTTGAAGACG TGGTTGGAAC GTCTTCTTTT TCCACGATGC TCCTCGTGGG |
| 8251 | TGGGGGTCCA TCTTTGGGAC CACTGTCGGC AGAGGCATCT TGAACGATAG |
| 8301 | CCTTTCCTTT ATCGCAATGA TGGCATTTGT AGGTGCCACC TTCCTTTTCT |
| 8351 | ACTGTCCTTT TGATGAAGTG ACAGATAGCT GGGCAATGGA ATCCGAGGAG |
| 8401 | GTTTCCCGAT ATTACCCTTT GTTGAAAAGT CTCAATAGCC CTTTGGTCTT |
| 8451 | CTGAGACTGT ATCTTTGATA TTCTTGGAGT AGACGAGAGT GTCGTGCTCC |
| 8501 | ACCATGTTAT CACATCAATC CACTTGCTTT GAAGACGTGG TTGGAACGTC |
| 8551 | TTCTTTTTCC ACGATGCTCC TCGTGGGTGG GGGTCCATCT TTGGGACCAC |
| 8601 | TGTCGGCAGA GGCATCTTGA ACGATAGCCT TTCCTTTATC GCAATGATGG |
| 8651 | CATTTGTAGG TGCCACCTTC CTTTTCTACT GTCCTTTTGA TGAAGTGACA |
| 8701 | GATAGCTGGG CAATGGAATC CGAGGAGGTT TCCCGATATT ACCCTTTGTT |
| 8751 | GAAAAGTCTC AATAGCCCTT TGGTCTTCTG AGACTGTATC TTTGATATTC |
| 8801 | TTGGAGTAGA CGAGAGTGTC GTGCTCCACC ATGTTGGCAA GCTGCTCTAG |
| 8851 | CCAATACGCA AACCGCCTCT CCCCGCGCGT TGGCCGATTC ATTAATGCAG |
| 8901 | CTGGCACGAC AGGTTTCCCG ACTGGAAAGC GGGCAGTGAG CGCAACGCAA |
| 8951 | TTAATGTGAG TTAGCTCACT CATTAGGCAC CCCAGGCTTT ACACTTTATG |
| 9001 | CTTCCGGCTC GTATGTTGTG TGGAATTGTG AGCGGATAAC AATTTCACAC |
| 9051 | AGGAAACAGC TATGACCATG ATTACGAATT CGAGCTCGGT ACCCGGGGAT |
| 9101 | CCTCTAGAGT CGACCTGCAG GCATGCAAGC TTGGCACTGG CCGTCGTTTT |
| 9151 | ACAACGTCGT GACTGGGAAA ACCCTGGCGT TACCCAACTT AATCGCCTTG |
| 9201 | CAGCACATCC CCCTTTCGCC AGCTGGCGTA ATAGCGAAGA GGCCCGCACC |
| 9251 | GATCGCCCTT CCCAACAGTT GCGCAGCCTG AATGGCGAAT GCTAGAGCAG |
| 9301 | CTTGAGCTTG GATCAGATTG TCGTTTCCCG CCTTCAGTTT AAACTATCAG |
| 9351 | TGTTTGACAG GATATATTGG CGGGTAAACC TAAGAGAAAA GAGCGTTTAT |
| 9401 | TAGAATAACG GATATTTAAA AGGGCGTGAA AAGGTTTATC CGTTCGTCCA |
| 9451 | TTTGTATGTG |

A DNA fragment containing a portion of the hsp70 intron fused to a gene fragment encoding sorbitol dehydrogenase (sdh) was synthesized by DNA 2.0 (Menlo Park, Calif.) and has the following nucleotide sequence.

| (SEQ ID NO: 2) | |
|---|---|
| 1 | TACGTATCTT GCTCGATGCC TTCTCCTAGT GTTGACCAGT GTTACTCACA |
| 51 | TAGTCTTTGC TCATTTCATT GTAATGCAGA TACCAAGCGG CAAAATGAGA |
| 101 | CTGGAAGACA AGGTCGCGAT CCTGACGGGC GCCGCAAGCG GCATCGGCGA |
| 151 | GGCGGTCGCA CAACGCTATC TGGACGAGGG CGCGCGCTGC GTGCTCGTCG |
| 201 | ACGTGAAGCC GGCAGGCGGC TCGCTCGCGC GGCTGATCGA GGCCAACCCG |
| 251 | GGCCGCGCGG TGGCCGTCAC GGCCGACGTC ACGCGTCGCG ACGACATCAC |
| 301 | GCGGATCGTC GCCACGGCGG TCGAGCGCTT CGGCGGCGTC GACATTCTGT |
| 351 | TCAACAACGC GGCGCTGTTC GACATGCGTC CGCTCCTCGA TGAATCCTGG |
| 401 | GACGTGTTCG ACCGGCTGTT CTCGGTCAAC GTGAAAGGGC TGTTCTTCCT |
| 451 | GATGCAGGCG GTTGCGCAAC GGATGGTCGA GCAGGGGCGC GGCGGCAAGA |
| 501 | TCGTCAACAT GTCGTCGCAG GCCGGCCGTC GCGGCGAGGC GCTCGTTTCG |
| 551 | CACTACTGCG CGACCAAGGC CGCGGTGATC AGCTATACGC AGTCGGCCGC |
| 601 | GCTCGCGCTC GCGCCGCACC GGATCAACGT GAACGGCATC GCGCCGGGCG |
| 651 | TGGTCGACAC GCCGATGTGG GAGCAGGTCG ATGCGCTGTT CGCGCGCTAC |
| 701 | GAGAACCGGC CGCTCGGCGA GAAGAAGCGG CTCGTCGGTG AAGCCGTGCC |
| 751 | GCTCGGCCGC ATGGGCGTGC CGGGCGACCT GACGGGCGCC GCGCTGTTCC |
| 801 | TCGCGTCGGC CGATGCCGAC TACATCACCG CCCAGACGTT GAACGTCGAT |
| 851 | GGCGGCAACT GGATGAGCTG ACTCGAGTGA ATTC |

### Example 4

### Transformation of Switchgrass with pMBXS323 Containing an Expression Cassette for the sdh Gene

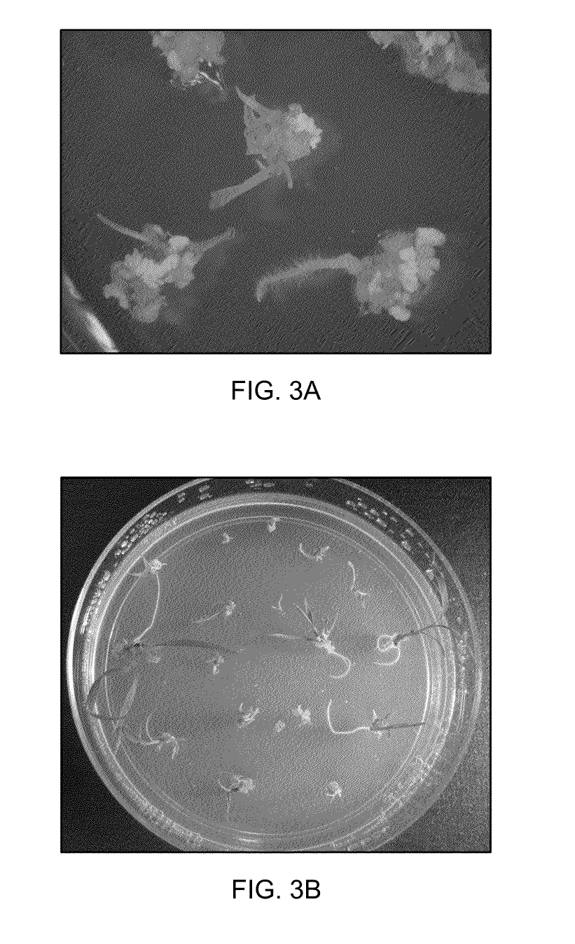

Agrobacterium-mediated transformation of switchgrass was performed as previously described (Somleva et al., 2002; Somleva, 2006). Highly embryogenic callus cultures were co-cultured with Agrobacterium tumifaciens strain AGL1 (Lazo et al., 1991) harboring pMBXS323 (FIG. 2) for three days in the dark at 28° C. The Agrobacterium treated cultures were incubated on a medium without selection for three to five days and then were transferred to medium containing sorbitol as the sole carbon source. After 4-6 wks of incubation in the dark at 28° C., 30-50% of the calli clumps showed the formation of new growth. These portions were carefully separated from the main callus and transferred to fresh selection medium for further callus proliferation. Upon transfer to regeneration medium containing sorbitol as the sole carbon source, these calli sectors developed green pigmentation within 3-5 days and eventually formed green adventitious shoots and emblings (somatic embryo derived plantlets) (FIGS. 3a-b).

Switchgrass transformation with plasmid pMBXS323 was also performed by particle bombardment procedures using a Biolistics PDS-1000/He apparatus (Bio-Rad Laboratories, Hercules, Calif., USA). Mature caryopses derived highly embryogenic callus cultures were targeted for the delivery of plasmid pMBXS323. DNA coating of gold particles (0.6 μm) and the subsequent delivery into target tissue were performed essentially as per the manufacturer's directions (Biolistic PDS-1000/He Particle delivery system, Biorad Laboratories, Hercules, Calif., USA).

The bombarded callus pieces were incubated for 3-5 days on a non-selection medium before transferring them to selection medium containing sorbitol as a sole carbon source.

Putative transgenic plantlets from both Agrobacterium-mediated and biolistic transformations were carefully removed from growth medium and roots were washed gently to remove agar. Healthy plants with a well developed root system were selected and transferred to a transplant tray filled with soil and incubated in plant growth chambers set at high humidity. All most all plants rapidly established roots and were moved to larger pots and grown in green house conditions.

### Example 5

### PCR Analysis of Transgenic Switchgrass Plants

Putative transgenic plants that were able to grow in the presence of sorbitol as the sole carbon source were analyzed for the sdh transgene using PCR on total nucleic acid extracts obtained from leaf tissues of soil grown plants.

For soil grown plants, total DNA was prepared with the Wizard® Genomic DNA Purification Kit (Promega Corporation, Madison, Wis.). PCR was performed with primers KMB 206 and KMB 207 designed to anneal to a portion of the SDH coding region and produce a 0.49 kb band.

| (SEQ ID NO: 3) | |
|---|---|
| KMB 206: | 5′ -TCGCACAACGCTATCTGGAC- 3′ |
| (SEQ ID NO: 4) | |
| KMB 207: | 5′ -GATGCCGTTCACGTTGATCC- 3′ |

PCR was performed using the following conditions: (a) 95° C. for 2 min (1 cycle); (b) 95° C. for 30 sec, 62° C. for 45 sec, 72° C. for 45 sec (35 cycles); 72° C. extension for 10 min.

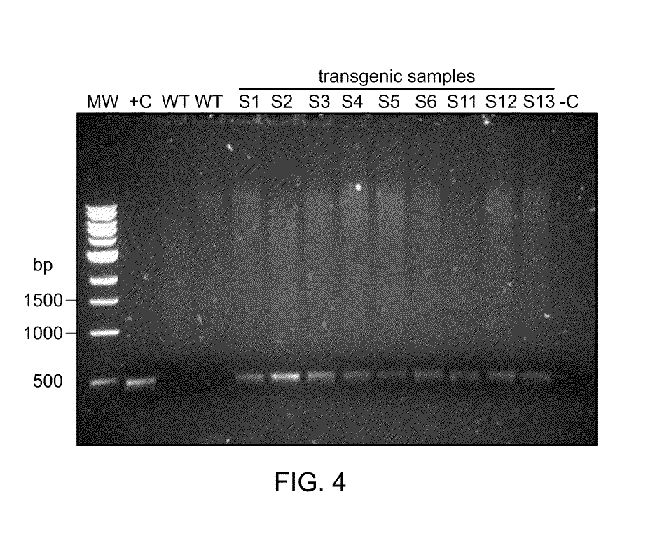

As shown in FIG. 4, a band of the correct size, 0.49 kb was present in the DNA of each of the putative transgenic lines tested (see S1-S6 and S11-S13) confirming the presence of the sorbitol dehydrogenase gene in these transgenic lines. This band was absent in the control lanes WT and WT.

### Example 6

### Southern Analysis of Transgenic Switchgrass Plants

Transgenic plants that were shown to be transformed with pMBXS323 using PCR to test for the presence of the sorbitol dehydrogenase gene (Example 5) were analyzed via Southern analysis to analyze independent transformation events and to determine the number of transgene copies present in each line. The Wizard® Genomic DNA Purification Kit (Promega Corporation, Madison, Wis.) was used for DNA extraction. For Southern analysis, 11 to 15 μg of total DNA was digested with the indicated restriction enzymes and blotted onto positively charged nylon membranes (Roche Molecular Biochemicals, Indianapolis). A digoxigenin-labeled hybridization probe for detection of the sdh gene was prepared with the DIG probe synthesis kit (Roche Molecular Biochemicals) using the following oligonucleotides:

| (SEQ ID NO: 3) | |
|---|---|
| KMB 206: | 5′ -TCGCACAACGCTATCTGGAC- 3′ |
| (SEQ ID NO: 4) | |
| KMB 207: | 5′ -GATGCCGTTCACGTTGATCC- 3′ |

PCR conditions for the amplifications including DIG-labeling were as follows: (a) 95° C. for 2 min (1 cycle); (b) 95° C. for 30 sec, 54° C. for 45 sec, 72° C. for 45 sec (30 cycles); 72° C. extension for 10 min.

Hybridization signals were detected with alkaline-phosphatase conjugated anti-digoxigenin antibody and chemoluminescent detection (CDP-Star, Roche Molecular Biochemicals).

Of 16 transgenic lines analyzed, eight independent transformation events were identified. Three events contained a single transgene copy insertion, four events contained two transgene copy insertions, and one event contained multiple inserted copies (>5) of the transgene. The observed phenotype of almost all of the plants isolated was comparable to wild-type.

### Example 7

### Use of Sorbitol Dehydrogenase as Selectable Marker in Transformation of Dicots

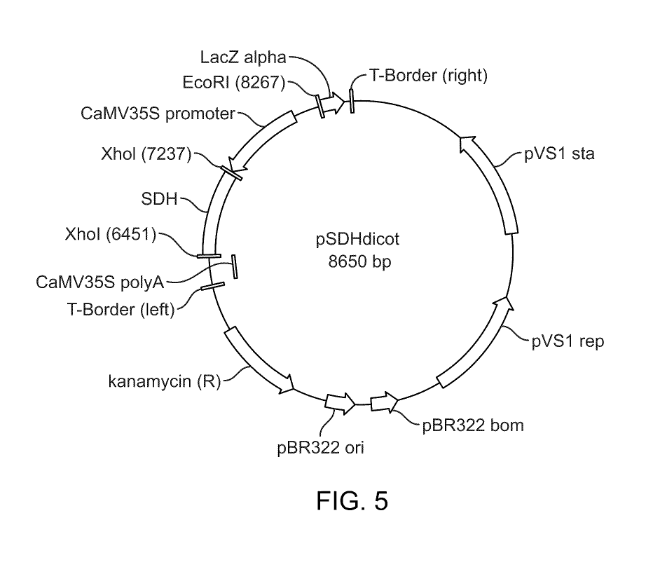

FIG. 5 shows a plant transformation vector (pSDH.dicot) that can enable the use of sorbitol dehydrogenase as a selectable marker in dicots. This pCAMBIA3300 based vector (Center for Application of Molecular Biology to International Agriculture, Canberra, Australia) contains an expression cassette for sorbitol dehydrogenase containing the CaMV35S promoter (Kay, R., et al., Science, 236: 1299-1302 (1987)), the sorbitol dehydrogenase gene (sdh) from Pseudomonas sp. KS-E1806, and the CaMV35S polyadenylation sequence (Odell, J., et al., Nature, 313(6005): 810-812 (1985)). The ATG of the sorbitol dehydrogenase coding sequence is preceded by the sequence “AAA”, an optimized Kozak sequence.

The nucleic sequence of plasmid pSDH.dicot is as follows:

| (SEQ ID NO: 5) | |
|---|---|
| 1 | CATGCCAACC ACAGGGTTCC CCTCGGGATC AAAGTACTTT GATCCAACCC |
| 51 | CTCCGCTGCT ATAGTGCAGT CGGCTTCTGA CGTTCAGTGC AGCCGTCTTC |
| 101 | TGAAAACGAC ATGTCGCACA AGTCCTAAGT TACGCGACAG GCTGCCGCCC |
| 151 | TGCCCTTTTC CTGGCGTTTT CTTGTCGCGT GTTTTAGTCG CATAAAGTAG |
| 201 | AATACTTGCG ACTAGAACCG GAGACATTAC GCCATGAACA AGAGCGCCGC |
| 251 | CGCTGGCCTG CTGGGCTATG CCCGCGTCAG CACCGACGAC CAGGACTTGA |
| 301 | CCAACCAACG GGCCGAACTG CACGCGGCCG GCTGCACCAA GCTGTTTTCC |
| 351 | GAGAAGATCA CCGGCACCAG GCGCGACCGC CCGGAGCTGG CCAGGATGCT |
| 401 | TGACCACCTA CGCCCTGGCG ACGTTGTGAC AGTGACCAGG CTAGACCGCC |
| 451 | TGGCCCGCAG CACCCGCGAC CTACTGGACA TTGCCGAGCG CATCCAGGAG |
| 501 | GCCGGCGCGG GCCTGCGTAG CCTGGCAGAG CCGTGGGCCG ACACCACCAC |
| 551 | GCCGGCCGGC CGCATGGTGT TGACCGTGTT CGCCGGCATT GCCGAGTTCG |
| 601 | AGCGTTCCCT AATCATCGAC CGCACCCGGA GCGGGCGCGA GGCCGCCAAG |
| 651 | GCCCGAGGCG TGAAGTTTGG CCCCCGCCCT ACCCTCACCC CGGCACAGAT |
| 701 | CGCGCACGCC CGCGAGCTGA TCGACCAGGA AGGCCGCACC GTGAAAGAGG |
| 751 | CGGCTGCACT GCTTGGCGTG CATCGCTCGA CCCTGTACCG CGCACTTGAG |
| 801 | CGCAGCGAGG AAGTGACGCC CACCGAGGCC AGGCGGCGCG GTGCCTTCCG |
| 851 | TGAGGACGCA TTGACCGAGG CCGACGCCCT GGCGGCCGCC GAGAATGAAC |
| 901 | GCCAAGAGGA ACAAGCATGA AACCGCACCA GGACGGCCAG GACGAACCGT |
| 951 | TTTTCATTAC CGAAGAGATC GAGGCGGAGA TGATCGCGGC CGGGTACGTG |
| 1001 | TTCGAGCCGC CCGCGCACGT CTCAACCGTG CGGCTGCATG AAATCCTGGC |
| 1051 | CGGTTTGTCT GATGCCAAGC TGGCGGCCTG GCCGGCCAGC TTGGCCGCTG |
| 1101 | AAGAAACCGA GCGCCGCCGT CTAAAAAGGT GATGTGTATT TGAGTAAAAC |
| 1151 | AGCTTGCGTC ATGCGGTCGC TGCGTATATG ATGCGATGAG TAAATAAACA |
| 1201 | AATACGCAAG GGGAACGCAT GAAGGTTATC GCTGTACTTA ACCAGAAAGG |
| 1251 | CGGGTCAGGC AAGACGACCA TCGCAACCCA TCTAGCCCGC GCCCTGCAAC |
| 1301 | TCGCCGGGGC CGATGTTCTG TTAGTCGATT CCGATCCCCA GGGCAGTGCC |
| 1351 | CGCGATTGGG CGGCCGTGCG GGAAGATCAA CCGCTAACCG TTGTCGGCAT |
| 1401 | CGACCGCCCG ACGATTGACC GCGACGTGAA GGCCATCGGC CGGCGCGACT |
| 1451 | TCGTAGTGAT CGACGGAGCG CCCCAGGCGG CGGACTTGGC TGTGTCCGCG |
| 1501 | ATCAAGGCAG CCGACTTCGT GCTGATTCCG GTGCAGCCAA GCCCTTACGA |
| 1551 | CATATGGGCC ACCGCCGACC TGGTGGAGCT GGTTAAGCAG CGCATTGAGG |
| 1601 | TCACGGATGG AAGGCTACAA GCGGCCTTTG TCGTGTCGCG GGCGATCAAA |
| 1651 | GGCACGCGCA TCGGCGGTGA GGTTGCCGAG GCGCTGGCCG GGTACGAGCT |
| 1701 | GCCCATTCTT GAGTCCCGTA TCACGCAGCG CGTGAGCTAC CCAGGCACTG |
| 1751 | CCGCCGCCGG CACAACCGTT CTTGAATCAG AACCCGAGGG CGACGCTGCC |
| 1801 | CGCGAGGTCC AGGCGCTGGC CGCTGAAATT AAATCAAAAC TCATTTGAGT |
| 1851 | TAATGAGGTA AAGAGAAAAT GAGCAAAAGC ACAAACACGC TAAGTGCCGG |
| 1901 | CCGTCCGAGC GCACGCAGCA GCAAGGCTGC AACGTTGGCC AGCCTGGCAG |
| 1951 | ACACGCCAGC CATGAAGCGG GTCAACTTTC AGTTGCCGGC GGAGGATCAC |
| 2001 | ACCAAGCTGA AGATGTACGC GGTACGCCAA GGCAAGACCA TTACCGAGCT |
| 2051 | GCTATCTGAA TACATCGCGC AGCTACCAGA GTAAATGAGC AAATGAATAA |
| 2101 | ATGAGTAGAT GAATTTTAGC GGCTAAAGGA GGCGGCATGG AAAATCAAGA |
| 2151 | ACAACCAGGC ACCGACGCCG TGGAATGCCC CATGTGTGGA GGAACGGGCG |
| 2201 | GTTGGCCAGG CGTAAGCGGC TGGGTTGTCT GCCGGCCCTG CAATGGCACT |
| 2251 | GGAACCCCCA AGCCCGAGGA ATCGGCGTGA CGGTCGCAAA CCATCCGGCC |
| 2301 | CGGTACAAAT CGGCGCGGCG CTGGGTGATG ACCTGGTGGA GAAGTTGAAG |
| 2351 | GCCGCGCAGG CCGCCCAGCG GCAACGCATC GAGGCAGAAG CACGCCCCGG |
| 2401 | TGAATCGTGG CAAGCGGCCG CTGATCGAAT CCGCAAAGAA TCCCGGCAAC |
| 2451 | CGCCGGCAGC CGGTGCGCCG TCGATTAGGA AGCCGCCCAA GGGCGACGAG |
| 2501 | CAACCAGATT TTTTCGTTCC GATGCTCTAT GACGTGGGCA CCCGCGATAG |
| 2551 | TCGCAGCATC ATGGACGTGG CCGTTTTCCG TCTGTCGAAG CGTGACCGAC |
| 2601 | GAGCTGGCGA GGTGATCCGC TACGAGCTTC CAGACGGGCA CGTAGAGGTT |
| 2651 | TCCGCAGGGC CGGCCGGCAT GGCCAGTGTG TGGGATTACG ACCTGGTACT |
| 2701 | GATGGCGGTT TCCCATCTAA CCGAATCCAT GAACCGATAC CGGGAAGGGA |
| 2751 | AGGGAGACAA GCCCGGCCGC GTGTTCCGTC CACACGTTGC GGACGTACTC |
| 2801 | AAGTTCTGCC GGCGAGCCGA TGGCGGAAAG CAGAAAGACG ACCTGGTAGA |
| 2851 | AACCTGCATT CGGTTAAACA CCACGCACGT TGCCATGCAG CGTACGAAGA |
| 2901 | AGGCCAAGAA CGGCCGCCTG GTGACGGTAT CCGAGGGTGA AGCCTTGATT |
| 2951 | AGCCGCTACA AGATCGTAAA GAGCGAAACC GGGCGGCCGG AGTACATCGA |
| 3001 | GATCGAGCTA GCTGATTGGA TGTACCGCGA GATCACAGAA GGCAAGAACC |
| 3051 | CGGACGTGCT GACGGTTCAC CCCGATTACT TTTTGATCGA TCCCGGCATC |
| 3101 | GGCCGTTTTC TCTACCGCCT GGCACGCCGC GCCGCAGGCA AGGCAGAAGC |
| 3151 | CAGATGGTTG TTCAAGACGA TCTACGAACG CAGTGGCAGC GCCGGAGAGT |
| 3201 | TCAAGAAGTT CTGTTTCACC GTGCGCAAGC TGATCGGGTC AAATGACCTG |
| 3251 | CCGGAGTACG ATTTGAAGGA GGAGGCGGGG CAGGCTGGCC CGATCCTAGT |
| 3301 | CATGCGCTAC CGCAACCTGA TCGAGGGCGA AGCATCCGCC GGTTCCTAAT |
| 3351 | GTACGGAGCA GATGCTAGGG CAAATTGCCC TAGCAGGGGA AAAAGGTCGA |
| 3401 | AAAGGTCTCT TTCCTGTGGA TAGCACGTAC ATTGGGAACC CAAAGCCGTA |
| 3451 | CATTGGGAAC CGGAACCCGT ACATTGGGAA CCCAAAGCCG TACATTGGGA |
| 3501 | ACCGGTCACA CATGTAAGTG ACTGATATAA AAGAGAAAAA AGGCGATTTT |
| 3551 | TCCGCCTAAA ACTCTTTAAA ACTTATTAAA ACTCTTAAAA CCCGCCTGGC |
| 3601 | CTGTGCATAA CTGTCTGGCC AGCGCACAGC CGAAGAGCTG CAAAAAGCGC |
| 3651 | CTACCCTTCG GTCGCTGCGC TCCCTACGCC CCGCCGCTTC GCGTCGGCCT |
| 3701 | ATCGCGGCCG CTGGCCGCTC AAAAATGGCT GGCCTACGGC CAGGCAATCT |
| 3751 | ACCAGGGCGC GGACAAGCCG CGCCGTCGCC ACTCGACCGC CGGCGCCCAC |
| 3801 | ATCAAGGCAC CCTGCCTCGC GCGTTTCGGT GATGACGGTG AAAACCTCTG |
| 3851 | ACACATGCAG CTCCCGGAGA CGGTCACAGC TTGTCTGTAA GCGGATGCCG |
| 3901 | GGAGCAGACA AGCCCGTCAG GGCGCGTCAG CGGGTGTTGG CGGGTGTCGG |
| 3951 | GGCGCAGCCA TGACCCAGTC ACGTAGCGAT AGCGGAGTGT ATACTGGCTT |
| 4001 | AACTATGCGG CATCAGAGCA GATTGTACTG AGAGTGCACC ATATGCGGTG |
| 4051 | TGAAATACCG CACAGATGCG TAAGGAGAAA ATACCGCATC AGGCGCTCTT |
| 4101 | CCGCTTCCTC GCTCACTGAC TCGCTGCGCT CGGTCGTTCG GCTGCGGCGA |
| 4151 | GCGGTATCAG CTCACTCAAA GGCGGTAATA CGGTTATCCA CAGAATCAGG |
| 4201 | GGATAACGCA GGAAAGAACA TGTGAGCAAA AGGCCAGCAA AAGGCCAGGA |
| 4251 | ACCGTAAAAA GGCCGCGTTG CTGGCGTTTT TCCATAGGCT CCGCCCCCCT |
| 4301 | GACGAGCATC ACAAAAATCG ACGCTCAAGT CAGAGGTGGC GAAACCCGAC |
| 4351 | AGGACTATAA AGATACCAGG CGTTTCCCCC TGGAAGCTCC CTCGTGCGCT |
| 4401 | CTCCTGTTCC GACCCTGCCG CTTACCGGAT ACCTGTCCGC CTTTCTCCCT |
| 4451 | TCGGGAAGCG TGGCGCTTTC TCATAGCTCA CGCTGTAGGT ATCTCAGTTC |
| 4501 | GGTGTAGGTC GTTCGCTCCA AGCTGGGCTG TGTGCACGAA CCCCCCGTTC |
| 4551 | AGCCCGACCG CTGCGCCTTA TCCGGTAACT ATCGTCTTGA GTCCAACCCG |
| 4601 | GTAAGACACG ACTTATCGCC ACTGGCAGCA GCCACTGGTA ACAGGATTAG |
| 4651 | CAGAGCGAGG TATGTAGGCG GTGCTACAGA GTTCTTGAAG TGGTGGCCTA |
| 4701 | ACTACGGCTA CACTAGAAGG ACAGTATTTG GTATCTGCGC TCTGCTGAAG |
| 4751 | CCAGTTACCT TCGGAAAAAG AGTTGGTAGC TCTTGATCCG GCAAACAAAC |
| 4801 | CACCGCTGGT AGCGGTGGTT TTTTTGTTTG CAAGCAGCAG ATTACGCGCA |
| 4851 | GAAAAAAAGG ATCTCAAGAA GATCCTTTGA TCTTTTCTAC GGGGTCTGAC |
| 4901 | GCTCAGTGGA ACGAAAACTC ACGTTAAGGG ATTTTGGTCA TGCATTCTAG |
| 4951 | GTACTAAAAC AATTCATCCA GTAAAATATA ATATTTTATT TTCTCCCAAT |
| 5001 | CAGGCTTGAT CCCCAGTAAG TCAAAAAATA GCTCGACATA CTGTTCTTCC |
| 5051 | CCGATATCCT CCCTGATCGA CCGGACGCAG AAGGCAATGT CATACCACTT |
| 5101 | GTCCGCCCTG CCGCTTCTCC CAAGATCAAT AAAGCCACTT ACTTTGCCAT |
| 5151 | CTTTCACAAA GATGTTGCTG TCTCCCAGGT CGCCGTGGGA AAAGACAAGT |
| 5201 | TCCTCTTCGG GCTTTTCCGT CTTTAAAAAA TCATACAGCT CGCGCGGATC |
| 5251 | TTTAAATGGA GTGTCTTCTT CCCAGTTTTC GCAATCCACA TCGGCCAGAT |
| 5301 | CGTTATTCAG TAAGTAATCC AATTCGGCTA AGCGGCTGTC TAAGCTATTC |
| 5351 | GTATAGGGAC AATCCGATAT GTCGATGGAG TGAAAGAGCC TGATGCACTC |
| 5401 | CGCATACAGC TCGATAATCT TTTCAGGGCT TTGTTCATCT TCATACTCTT |
| 5451 | CCGAGCAAAG GACGCCATCG GCCTCACTCA TGAGCAGATT GCTCCAGCCA |
| 5501 | TCATGCCGTT CAAAGTGCAG GACCTTTGGA ACAGGCAGCT TTCCTTCCAG |
| 5551 | CCATAGCATC ATGTCCTTTT CCCGTTCCAC ATCATAGGTG GTCCCTTTAT |
| 5601 | ACCGGCTGTC CGTCATTTTT AAATATAGGT TTTCATTTTC TCCCACCAGC |
| 5651 | TTATATACCT TAGCAGGAGA CATTCCTTCC GTATCTTTTA CGCAGCGGTA |
| 5701 | TTTTTCGATC AGTTTTTTCA ATTCCGGTGA TATTCTCATT TTAGCCATTT |
| 5751 | ATTATTTCCT TCCTCTTTTC TACAGTATTT AAAGATACCC CAAGAAGCTA |
| 5801 | ATTATAACAA GACGAACTCC AATTCACTGT TCCTTGCATT CTAAAACCTT |
| 5851 | AAATACCAGA AAACAGCTTT TTCAAAGTTG TTTTCAAAGT TGGCGTATAA |
| 5901 | CATAGTATCG ACGGAGCCGA TTTTGAAACC GCGGTGATCA CAGGCAGCAA |
| 5951 | CGCTCTGTCA TCGTTACAAT CAACATGCTA CCCTCCGCGA GATCATCCGT |
| 6001 | GTTTCAAACC CGGCAGCTTA GTTGCCGTTC TTCCGAATAG CATCGGTAAC |
| 6051 | ATGAGCAAAG TCTGCCGCCT TACAACGGCT CTCCCGCTGA CGCCGTCCCG |
| 6101 | GACTGATGGG CTGCCTGTAT CGAGTGGTGA TTTTGTGCCG AGCTGCCGGT |
| 6151 | CGGGGAGCTG TTGGCTGGCT GGTGGCAGGA TATATTGTGG TGTAAACAAA |
| 6201 | TTGACGCTTA GACAACTTAA TAACACATTG CGGACGTTTT TAATGTACTG |
| 6251 | AATTAACGCC GAATTAATTC GGGGGATCTG GATTTTAGTA CTGGATTTTG |
| 6301 | GTTTTAGGAA TTAGAAATTT TATTGATAGA AGTATTTTAC AAATACAAAT |
| 6351 | ACATACTAAG GGTTTCTTAT ATGCTCAACA CATGAGCGAA ACCCTATAGG |
| 6401 | AACCCTAATT CCCTTATCTG GGAACTACTC ACACATTATT ATGGAGAAAC |
| 6451 | TCGAGTCAGC TCATCCAGTT GACGCCATCG ACGTTCAACG TCTGGGCGGT |
| 6501 | GATGTAGTCG GCATCGGCCG ACGCGAGGAA CAGCGCGGCG CCCGTCAGGT |
| 6551 | CGCCCGGCAC GCCCATGCGG CCGAGCGGCA CGGCTTCACC GACGAGCCGC |
| 6601 | TTCTTCTCGC CGAGCGGCCG GTTCTCGTAG CGCGCGAACA GCGCATCGAC |
| 6651 | CTGCTCCCAC ATCGGCGTGT CGACCACGCC CGGCGCGATG CCGTTCACGT |
| 6701 | TGATCCGGTG CGGCGCGAGC GCGAGCGCGG CCGACTGCGT ATAGCTGATC |
| 6751 | ACCGCGGCCT TGGTCGCGCA GTAGTGCGAA ACGAGCGCCT CGCCGCGACG |
| 6801 | GCCGGCCTGC GACGACATGT TGACGATCTT GCCGCCGCGC CCCTGCTCGA |
| 6851 | CCATCCGTTG CGCAACCGCC TGCATCAGGA AGAACAGCCC TTTCACGTTG |
| 6901 | ACCGAGAACA GCCGGTCGAA CACGTCCCAG GATTCATCGA GGAGCGGACG |
| 6951 | CATGTCGAAC AGCGCCGCGT TGTTGAACAG AATGTCGACG CCGCCGAAGC |
| 7001 | GCTCGACCGC CGTGGCGACG ATCCGCGTGA TGTCGTCGCG ACGCGTGACG |
| 7051 | TCGGCCGTGA CGGCCACCGC GCGGCCCGGG TTGGCCTCGA TCAGCCGCGC |
| 7101 | GAGCGAGCCG CCTGCCGGCT TCACGTCGAC GAGCACGCAG CGCGCGCCCT |
| 7151 | CGTCCAGATA GCGTTGTGCG ACCGCCTCGC CGATGCCGCT TGCGGCGCCC |
| 7201 | GTCAGGATCG CGACCTTGTC TTCCAGTCTC ATTTTCTCGA GAGAGATAGA |
| 7251 | TTTGTAGAGA GAGACTGGTG ATTTCAGCGT GTCCTCTCCA AATGAAATGA |
| 7301 | ACTTCCTTAT ATAGAGGAAG GTCTTGCGAA GGATAGTGGG ATTGTGCGTC |
| 7351 | ATCCCTTACG TCAGTGGAGA TATCACATCA ATCCACTTGC TTTGAAGACG |
| 7401 | TGGTTGGAAC GTCTTCTTTT TCCACGATGC TCCTCGTGGG TGGGGGTCCA |
| 7451 | TCTTTGGGAC CACTGTCGGC AGAGGCATCT TGAACGATAG CCTTTCCTTT |
| 7501 | ATCGCAATGA TGGCATTTGT AGGTGCCACC TTCCTTTTCT ACTGTCCTTT |
| 7551 | TGATGAAGTG ACAGATAGCT GGGCAATGGA ATCCGAGGAG GTTTCCCGAT |
| 7601 | ATTACCCTTT GTTGAAAAGT CTCAATAGCC CTTTGGTCTT CTGAGACTGT |
| 7651 | ATCTTTGATA TTCTTGGAGT AGACGAGAGT GTCGTGCTCC ACCATGTTAT |
| 7701 | CACATCAATC CACTTGCTTT GAAGACGTGG TTGGAACGTC TTCTTTTTCC |
| 7751 | ACGATGCTCC TCGTGGGTGG GGGTCCATCT TTGGGACCAC TGTCGGCAGA |
| 7801 | GGCATCTTGA ACGATAGCCT TTCCTTTATC GCAATGATGG CATTTGTAGG |
| 7851 | TGCCACCTTC CTTTTCTACT GTCCTTTTGA TGAAGTGACA GATAGCTGGG |
| 7901 | CAATGGAATC CGAGGAGGTT TCCCGATATT ACCCTTTGTT GAAAAGTCTC |
| 7951 | AATAGCCCTT TGGTCTTCTG AGACTGTATC TTTGATATTC TTGGAGTAGA |
| 8001 | CGAGAGTGTC GTGCTCCACC ATGTTGGCAA GCTGCTCTAG CCAATACGCA |
| 8051 | AACCGCCTCT CCCCGCGCGT TGGCCGATTC ATTAATGCAG CTGGCACGAC |
| 8101 | AGGTTTCCCG ACTGGAAAGC GGGCAGTGAG CGCAACGCAA TTAATGTGAG |
| 8151 | TTAGCTCACT CATTAGGCAC CCCAGGCTTT ACACTTTATG CTTCCGGCTC |
| 8201 | GTATGTTGTG TGGAATTGTG AGCGGATAAC AATTTCACAC AGGAAACAGC |
| 8251 | TATGACCATG ATTACGAATT CGAGCTCGGT ACCCGGGGAT CCTCTAGAGT |
| 8301 | CGACCTGCAG GCATGCAAGC TTGGCACTGG CCGTCGTTTT ACAACGTCGT |
| 8351 | GACTGGGAAA ACCCTGGCGT TACCCAACTT AATCGCCTTG CAGCACATCC |
| 8401 | CCCTTTCGCC AGCTGGCGTA ATAGCGAAGA GGCCCGCACC GATCGCCCTT |
| 8451 | CCCAACAGTT GCGCAGCCTG AATGGCGAAT GCTAGAGCAG CTTGAGCTTG |
| 8501 | GATCAGATTG TCGTTTCCCG CCTTCAGTTT AAACTATCAG TGTTTGACAG |
| 8551 | GATATATTGG CGGGTAAACC TAAGAGAAAA GAGCGTTTAT TAGAATAACG |
| 8601 | GATATTTAAA AGGGCGTGAA AAGGTTTATC CGTTCGTCCA TTTGTATGTG |

### Example 8

### Callus Induction and Shoot Regeneration from Tobacco Leaves in Tissue Culture in the Presence of Sorbitol

To test whether sorbitol dehydrogenase can be used as a positive selection marker in tobacco, pieces of tobacco leaves were tested on media containing different sugars as a sole carbon source.

Sterile grown tobacco leaves were cut into pieces of approximately 0.5-1 cm². Leaf pieces were transferred onto MS media containing minimal organics (MSP002 from Caisson Laboratories, North Logan, Utah, USA), 1 mg/L 6-BAP (6-benzylaminopurine) in 1N NaOH, 100 ug/L NAA (α-naphtahalene acetic acid), and the following carbon sources: no sugar; sorbitol, (16 g/L); fructose, (15.8 g/L); sucrose (30 g/L). Explants were maintained in tissue culture for 4 weeks with the following light cycle: 16 hrs in the light at 23° C.; 8 hrs in the dark at 20° C.; relative humidity approximately 45%.

Inhibited callus generation and inhibited shoot regeneration on sorbitol indicated that these cultures could not use sorbitol as a sole carbon source either due to a lack of, or insufficient amounts of sorbitol dehydrogenase. Callus induction and shoot regeneration on fructose indicated the ability of tobacco to use fructose as a sole carbon source. These results indicate that the sorbitol dehydrogenase marker and sorbitol can be used for selection of tobacco leaf cultures in both nuclear and plastid transformation procedures.

### Example 9

### Use of Sorbitol Dehydrogenase as a Selectable Marker in Plastid Transformation

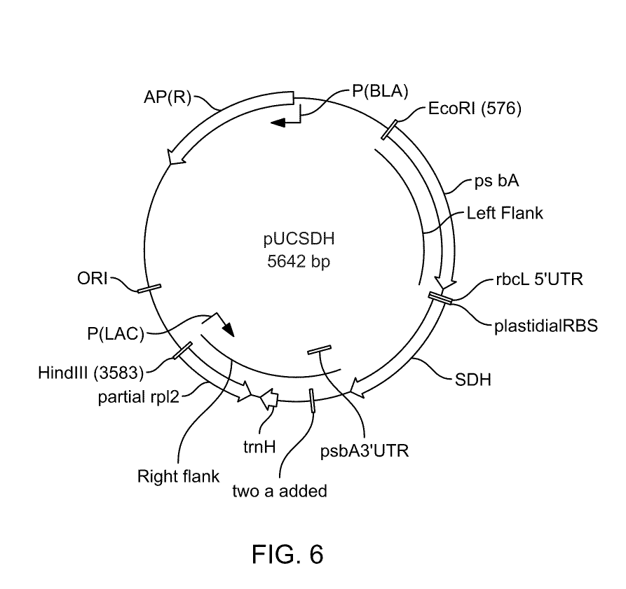

To test sorbitol dehydrogenase as a selectable marker in plastid transformation, plasmid pUCSDH (FIG. 6) was designed. The gene encoding sorbitol dehydrogenase (sdh) is flanked by sequences of the tobacco plastid genome to initiate homologous recombination between the psbA structural gene (left flank) and the psbA 3′ UTR, (right flank) in the plastid genome (FIG. 6). The sequence for plasmid pUCSDH is as follows:

| (SEQ ID NO: 6) | |
|---|---|
| 1 | TGAAGCATT TATCAGGGTT ATTGTCTCAT GAGCGGATAC ATATTTGAAT |
| 51 | GTATTTAGAA AAATAAACAA ATAGGGGTTC CGCGCACATT TCCCCGAAAA |
| 101 | GTGCCACCTG ACGTCTAAGA AACCATTATT ATCATGACAT TAACCTATAA |
| 151 | AAATAGGCGT ATCACGAGGC CCTTTCGTCT CGCGCGTTTC GGTGATGACG |
| 201 | TGAAAACCT CTGACACATG CAGCTCCCGG AGACGGTCAC AGCTTGTCTG |
| 251 | AAGCGGATG CCGGGAGCAG ACAAGCCCGT CAGGGCGCGT CAGCGGGTGT |
| 301 | GGCGGGTGT CGGGGCTGGC TTAACTATGC GGCATCAGAG CAGATTGTAC |
| 351 | AGAGTGCA CCATATGCGG TGTGAAATAC CGCACAGATG CGTAAGGAGA |
| 401 | AATACCGCA TCAGGCGCCA TTCGCCATTC AGGCTGCGCA ACTGTTGGGA |
| 451 | AGGGCGATCG GTGCGGGCCT CTTCGCTATT ACGCAAGCTG GCGAAAGGGG |
| 501 | GATGTGCTGC AAGGCGATTA AGTTGGGTAA CGCCAGGGTT TTCCCAGTCA |
| 551 | CGACGTTGTA AAACGACGGC CAGTGAATTC ATGACTGCAA TTTTAGAGAG |
| 601 | ACGCGAAAGC GAAAGCCTAT GGGGTCGCTT CTGTAACTGG ATAACTAGCA |
| 651 | CTGAAAACCG TCTTTACATT GGATGGTTTG GTGTTTTGAT GATCCCTACC |
| 701 | TTATTGACGG CAACTTCTGT ATTTATTATT GCCTTCATTG CTGCTCCTCC |
| 751 | AGTAGACATT GATGGTATTC GTGAACCTGT TTCAGGGTCT CTACTTTACG |
| 801 | GAAACAATAT TATTTCCGGT GCCATTATTC CTACTTCTGC AGCTATAGGT |
| 851 | TTACATTTTT ACCCAATCTG GGAAGCGGCA TCCGTTGATG AATGGTTATA |
| 901 | CAACGGTGGT CCTTATGAAC TAATTGTTCT ACACTTCTTA CTTGGCGTAG |
| 951 | CTTGTTACAT GGGTCGTGAG TGGGAGCTTA GTTTCCGTCT GGGTATGCGA |
| 1001 | CCTTGGATTG CTGTTGCATA TTCAGCTCCT GTTGCAGCTG CTACCGCAGT |
| 1051 | TTTCTTGATC TACCCAATTG GTCAAGGAAG TTTTTCTGAT GGTATGCCTC |
| 1101 | TAGGAATCTC TGGTACTTTC AATTTCATGA TTGTATTCCA GGCTGAGCAC |
| 1151 | AACATCCTTA TGCACCCATT TCACATGTTA GGCGTAGCTG GTGTATTCGG |
| 1201 | CGGCTCCCTA TTCAGTGCTA TGCATGGTTC CTTGGTAACT TCTAGTTTGA |
| 1251 | TCAGGGAAAC CACAGAAAAT GAATCTGCTA ATGAAGGTTA CAGATTCGGT |
| 1301 | CAAGAGGAAG AAACTTATAA CATCGTAGCC GCTCATGGTT ATTTTGGCCG |
| 1351 | ATTGATCTTC CAATATGCTA GTTTCAACAA CTCTCGTTCG TTACACTTCT |
| 1401 | TCCTAGCTGC TTGGCCTGTA GTAGGTATCT GGTTTACCGC TTTAGGTATC |
| 1451 | AGCACTATGG CTTTCAACCT AAATGGTTTC AATTTCAACC AATCTGTAGT |
| 1501 | TGACAGTCAA GGCCGTGTAA TTAATACTTG GGCTGATATC ATTAACCGTG |
| 1551 | CTAACCTTGG TATGGAAGTT ATGCATGAAC GTAATGCTCA CAACTTCCCT |
| 1601 | CTAGACCTAG CTGCTATCGA AGCTCCATCT ACAAATGGAT AAGTCGACAA |
| 1651 | GTGTTTGCGG CCGCGAGCTC GGACTCGAGT TTGGATCCAA TCGATACAAG |
| 1701 | TGAGTTGTAG GGAGGGAACC ATGAGACTGG AAGACAAGGT CGCGATCCTG |
| 1751 | ACGGGCGCCG CAAGCGGCAT CGGCGAGGCG GTCGCACAAC GCTATCTGGA |
| 1801 | CGAGGGCGCG CGCTGCGTGC TCGTCGATGT GAAGCCGGCA GGCGGCTCGC |
| 1851 | TCGCGCGGCT GATCGAGGCC AACCCGGGCC GCGCGGTGGC CGTCACGGCC |
| 1901 | GACGTCACGC GTCGCGACGA CATCACGCGG ATCGTCGCCA CGGCGGTCGA |
| 1951 | GCGCTTCGGC GGCGTTGACA TTCTGTTCAA CAACGCGGCG CTGTTCGACA |
| 2001 | TGCGTCCGCT CCTCGATGAA TCCTGGGACG TGTTCGACCG GCTGTTCTCG |
| 2051 | GTCAACGTGA AAGGGCTGTT CTTCCTGATG CAGGCGGTTG CGCAACGGAT |
| 2101 | GGTCGAGCAG GGGCGCGGCG GCAAGATCGT CAACATGTCG TCGCAGGCCG |
| 2151 | GCCGTCGCGG CGAGGCGCTC GTTTCGCACT ACTGCGCGAC CAAGGCCGCG |
| 2201 | GTGATCAGCT ATACGCAGTC GGCCGCGCTC GCGCTCGCGC CGCACCGGAT |
| 2251 | CAACGTGAAC GGCATCGCGC CGGGCGTGGT CGATACGCCG ATGTGGGAGC |
| 2301 | AGGTCGATGC GCTGTTCGCG CGCTACGAGA ACCGGCCGCT CGGCGAGAAG |
| 2351 | AAGCGGCTCG TCGGTGAAGC CGTGCCGCTC GGCCGCATGG GCGTGCCGGG |
| 2401 | CGACCTGACG GGCGCCGCGC TGTTCCTCGC GTCGGCCGAT GCCGACTACA |
| 2451 | TCACCGCCCA GACGTTGAAC GTCGATGGCG GCAACTGGAT GAGCTGAATC |
| 2501 | TAAGCCGAAT TGGGCCTAGT CTATAGGAGG TTTTGAAAAG AAAGGAGCAA |
| 2551 | TAATCATTTT CTTGTTCTAT CAAGAGGGTG CTATTGCTCC TTTCTTTTTT |
| 2601 | TCTTTTTATT TATTTACTAG TATTTTACTT ACATAGACTT TTTTGTTTAC |
| 2651 | ATTATAGAAA AAGAAGGAGA GGTTATTTTC TTGCATTTAT TCATGATTGA |
| 2701 | GTATTCTATT TTGATTTTGT ATTTGTTTAA AATTGTAGAA ATAGAACTTG |
| 2751 | TTTCTCTTCT TGCTAATGTT ACTATATCTT TTTGATTTTT TTTTTCCAAA |
| 2801 | AAAAAAATCA AATTTTGACT TCTTCTTATC TCTTATCTTT GAATATCTCT |
| 2851 | TATCTTTGAA ATAATAATAT CATTGAAATA AGAAAGAAGA GCTATATTCG |
| 2901 | AACTTGAATC TTTTGTTTTC TAATTTAAAT AATGTAAAAA CGGAATGTAA |
| 2951 | GTAGGCGAGG GGGCGGATGT AGCCAAGTGG ATCAAGGCAG TGGATTGTGA |
| 3001 | ATCCACCATG CGCGGGTTCA ATTCCCGTCG TTCGCCCATA ATTACTCCTA |
| 3051 | TTTTTTTTTT TTTTGTAAAA ACGAAGAATT TAATTCGATT TTCTCTCCTA |
| 3101 | TTTACTACGG CGACGAAGAA TCAAATTATC ACTATATTTA TTCCTTTTTC |
| 3151 | TACTTCTTCT TCCAAGTGCA GGATAACCCC AAGGGGTTGT GGGTTTTTTT |
| 3201 | CTACCAATTG GGGCTCTCCC TTCACCACCC CCATGGGGAT GGTCTACAGG |
| 3251 | GTTCATAACT ACTCCTCTTA CTACAGGACG CTTACCTAGC CAACGCTTAG |
| 3301 | ATCCGGCTCT ACCCAAACTT TTCTGGTTCA CCCCAACATT CCCCACTTGT |
| 3351 | CCGACTGTTG CTGAGCAGTT TTTGGATATC AAACGGACCT CCCCAGAAGG |
| 3401 | TAATTTTAAT GTGGCCGATT TCCCCTCTTT TGCAATCAGT TTCGCTACAG |
| 3451 | CACCCGCTGC TCTAGCTAAT TGTCCACCCT TTCCAAGTGT GATTTCTATG |
| 3501 | TTATGTATGG CCGTGCCTAA GGGCATATCG GTTGAAGTAG ATTCTTCTTT |
| 3551 | TGATCAATCA AAACCCCTTC CCAAACTGTA CAAGCTTGGC GTAATCATGG |
| 3601 | TCATAGCTGT TTCCTGTGTG AAATTGTTAT CCGCTCACAA TTCCACACAA |
| 3651 | CATACGAGCC GGAAGCATAA AGTGTAAAGC CTGGGGTGCC TAATGAGTGA |
| 3701 | GCTAACTCAC ATTAATTGCG TTGCGCTCAC TGCCCGCTTT CCAGTCGGGA |
| 3751 | AACCTGTCGT GCCAGCTGCA TTAATGAATC GGCCAACGCG CGGGGAGAGG |
| 3801 | CGGTTTGCGT ATTGGGCGCT CTTCCGCTTC CTCGCTCACT GACTCGCTGC |
| 3851 | GCTCGGTCGT TCGGCTGCGG CGAGCGGTAT CAGCTCACTC AAAGGCGGTA |
| 3901 | ATACGGTTAT CCACAGAATC AGGGGATAAC GCAGGAAAGA ACATGTGAGC |
| 3951 | AAAAGGCCAG CAAAAGGCCA GGAACCGTAA AAAGGCCGCG TTGCTGGCGT |
| 4001 | TTTTCCATAG GCTCCGCCCC CCTGACGAGC ATCACAAAAA TCGACGCTCA |
| 4051 | AGTCAGAGGT GGCGAAACCC GACAGGACTA TAAAGATACC AGGCGTTTCC |
| 4101 | CCCTGGAAGC TCCCTCGTGC GCTCTCCTGT TCCGACCCTG CCGCTTACCG |
| 4151 | GATACCTGTC CGCCTTTCTC CCTTCGGGAA GCGTGGCGCT TTCTCATAGC |
| 4201 | TCACGCTGTA GGTATCTCAG TTCGGTGTAG GTCGTTCGCT CCAAGCTGGG |
| 4251 | CTGTGTGCAC GAACCCCCCG TTCAGCCCGA CCGCTGCGCC TTATCCGGTA |
| 4301 | ACTATCGTCT TGAGTCCAAC CCGGTAAGAC ACGACTTATC GCCACTGGCA |
| 4351 | GCAGCCACTG GTAACAGGAT TAGCAGAGCG AGGTATGTAG GCGGTGCTAC |
| 4401 | AGAGTTCTTG AAGTGGTGGC CTAACTACGG CTACACTAGA AGGACAGTAT |
| 4451 | TTGGTATCTG CGCTCTGCTG AAGCCAGTTA CCTTCGGAAA AAGAGTTGGT |
| 4501 | AGCTCTTGAT CCGGCAAACA AACCACCGCT GGTAGCGGTG GTTTTTTTGT |
| 4551 | TTGCAAGCAG CAGATTACGC GCAGAAAAAA AGGATCTCAA GAAGATCCTT |
| 4601 | TGATCTTTTC TACGGGGTCT GACGCTCAGT GGAACGAAAA CTCACGTTAA |
| 4651 | GGGATTTTGG TCATGAGATT ATCAAAAAGG ATCTTCACCT AGATCCTTTT |
| 4701 | AAATTAAAAA TGAAGTTTTA AATCAATCTA AAGTATATAT GAGTAAACTT |
| 4751 | GGTCTGACAG TTACCAATGC TTAATCAGTG AGGCACCTAT CTCAGCGATC |
| 4801 | TGTCTATTTC GTTCATCCAT AGTTGCCTGA CTCCCCGTCG TGTAGATAAC |
| 4851 | TACGATACGG GAGGGCTTAC CATCTGGCCC CAGTGCTGCA ATGATACCGC |
| 4901 | GAGACCCACG CTCACCGGCT CCAGATTTAT CAGCAATAAA CCAGCCAGCC |
| 4951 | GGAAGGGCCG AGCGCAGAAG TGGTCCTGCA ACTTTATCCG CCTCCATCCA |
| 5001 | GTCTATTAAT TGTTGCCGGG AAGCTAGAGT AAGTAGTTCG CCAGTTAATA |
| 5051 | GTTTGCGCAA CGTTGTTGCC ATTGCTACAG GCATCGTGGT GTCACGCTCG |
| 5101 | TCGTTTGGTA TGGCTTCATT CAGCTCCGGT TCCCAACGAT CAAGGCGAGT |
| 5151 | TACATGATCC CCCATGTTGT GCAAAAAAGC GGTTAGCTCC TTCGGTCCTC |
| 5201 | CGATCGTTGT CAGAAGTAAG TTGGCCGCAG TGTTATCACT CATGGTTATG |
| 5251 | GCAGCACTGC ATAATTCTCT TACTGTCATG CCATCCGTAA GATGCTTTTC |
| 5301 | TGTGACTGGT GAGTACTCAA CCAAGTCATT CTGAGAATAG TGTATGCGGC |
| 5351 | GACCGAGTTG CTCTTGCCCG GCGTCAATAC GGGATAATAC CGCGCCACAT |
| 5401 | AGCAGAACTT TAAAAGTGCT CATCATTGGA AAACGTTCTT CGGGGCGAAA |
| 5451 | ACTCTCAAGG ATCTTACCGC TGTTGAGATC CAGTTCGATG TAACCCACTC |
| 5501 | GTGCACCCAA CTGATCTTCA GCATCTTTTA CTTTCACCAG CGTTTCTGGG |
| 5551 | TGAGCAAAAA CAGGAAGGCA AAATGCCGCA AAAAAGGGAA TAAGGGCGAC |
| 5601 | ACGGAAATGT TGAATACTCA TACTCTTCCT TTTTCAATAT TA |

Plastid transformation of tobacco can be performed as follows. Seeds of tobacco (Nicotiana tabacum L. cv. ‘Petite Havana SR1’) are obtained from Lehle Seeds (Round Rock, Tex., USA). Plants in tissue culture are grown (16 h light period, 20 to 30 μmol photons m⁻² s⁻¹, 23° C.; 8 h dark period, 20° C.) on Murashige and Skoog medium (Murashige et al., 1962) containing 3% (w/v) sucrose. Plastid transformation is performed using a PDS 1000 System (BIORAD, Hercules, Calif., USA) and 0.6 μm gold particles as previously described (Svab, Z., P. et al., PNAS, 87(21): 8526-8530 (1990)).

Aseptically grown tobacco leaves 3-5 cm in length are placed leaf abaxial side up (“upside down”) on RMOP media (Daniell, H. “Transformation and Foreign Gene Expression in Plants Mediated by Microprojectile Bombardment” In Methods in Molecular Biology. R. Tuan. Totowa, N. J., Humana Press Inc. 62: 463-489 (1997)) for bombardment. After two days incubation in the dark, bombarded leaves are cut into pieces of 1 cm² and transferred to fresh RMOP media containing 1.6% sorbitol (w/v). Regenerating green shoots are transferred to Murashige and Skoog medium (Murashige, T. and F. Skoog, Physiol. Plant, 15: 473-497 (1962)) containing 1.6% (w/v) sorbitol for rooting. Leaves of regenerated plants are used for additional regeneration cycles (typically 1 to 3 cycles) to achieve homoplasmy.

Once transferred to soil, plants are grown in growth chambers (16 h light period, 40 to 80 μmol photons m⁻² s⁻¹, 23° C.; 8 h dark period, 20° C.) or in a greenhouse with supplemental lighting (16 h light period, minimum 150 μmol photons m⁻² s⁻¹, 23-25° C.; 8 h dark period, 20-22° C.).

Collectively, these results demonstrate that sorbitol dehydrogenase can be used as a selectable marker in both nuclear and plastid plant transformations.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A transgenic plant or transgenic plant cell comprising one or more heterologous nucleic acids encoding a polypeptide having sorbitol dehydrogenase activity and a second polypeptide, wherein the transgenic plant or transgenic plant cell expresses an effective amount of the polypeptide having sorbitol dehydrogenase activity for the transgenic plant or transgenic plant cell to grow using sorbitol as a sole source of carbon.

2. The transgenic plant or transgenic plant cell of claim 1 wherein the transgenic plant or plant cell is selected from the group consisting of Brassica family, industrial oilseeds, Arabidopsis thaHana* algae, soybean, cottonseed, sunflower, palm, coconut, rice, safflower, peanut, mustards, silage corn, alfalfa, switchgrass, miscanthus, sorghum, tobacco, sugarcane and flax.

3. The transgenic plant or transgenic plant cell of claim 2 wherein the Brassica family includes members selected from the group consisting of napus, rappa, sp. carinata and juncea.

4. The transgenic plant or transgenic plant cell of claim 2 wherein the industrial oilseeds are selected from the group consisting of Camelina sativa, Crambe, Jatropha, and castor.

5. The transgenic plant or transgenic plant cell of claim 1 wherein the transgenic plant or plant cell is a dicotyledon.

6. The transgenic plant or transgenic plant cell of claim 1 wherein the transgenic plant or plant cell is a monocotyledon.

7. The transgenic plant or transgenic plant cell of claim 1 wherein the heterologous nucleic acid is transcribed in the nucleus.

8. The transgenic plant or transgenic plant cell of claim 1 wherein the heterologous nucleic acid is transcribed in a plastid.

9. The transgenic plant or transgenic plant cell of claim 9 wherein the plastid is selected from the group consisting of chloroplasts, etioplasts, chromoplast, leucoplasts, amyloplasts, statoliths, elaioplasts, proteinoplasts and combinations thereof.

10. A method of culturing a transgenic plant comprising transforming a plant having no endogenous sorbitol dehydrogenase activity, or insufficient amounts of sorbitol dehyrogenase activity to allow growth on sorbitol, with a heterologous nucleic acid encoding a polypeptide having sorbitol dehydrogenase activity, wherein the transformed plant expresses an effective amount of the polypeptide having sorbitol dehydrogenase activity for the transformed plant to grow using sorbitol as a sole source of carbon, and culturing the transgenic plant using sorbitol as the sole source of carbon.

11. The method of claim 10 wherein the transgenic plant is a dicotyledon.

12. The method of claim 10 wherein the transgenic plant is a monocotyledon.

13. The method of claim 12 wherein the transgenic plant is switchgrass, sugarcane, sorghum, corn or miscanthus.

14. A nucleic acid construct comprising a nucleic acid according to SEQ ID NO:1, 2, 5 or 6 or a complement thereof.
